SEQUENCE LISTING (31 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, unidentified
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis D virus
RKLKKKIKKL

SEQ ID NO: 15 - protein, 10 aa, Mus sp.
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 20 - RNA, 23 nt, synthetic construct
  source 1..23: mol_type = other RNA
cttcgccttctcgcccccgccgg

SEQ ID NO: 21 - RNA, 23 nt, synthetic construct
  source 1..23: mol_type = other RNA
tgtcccgccatggggaaggaagg

SEQ ID NO: 22 - RNA, 20 nt, synthetic construct
  source 1..20: mol_type = other RNA
atgctctccaggttgcctct

SEQ ID NO: 23 - RNA, 20 nt, synthetic construct
  source 1..20: mol_type = other RNA
tcctcgaagttcatcacccg

SEQ ID NO: 24 - RNA, 19 nt, synthetic construct
  source 1..19: mol_type = other RNA
catgatatagacgttgtgg

SEQ ID NO: 25 - DNA, 20 bp, synthetic construct
  source 1..20: mol_type = other DNA
ggcaaggaacttgatgcacg

SEQ ID NO: 26 - DNA, 20 bp, synthetic construct
  source 1..20: mol_type = other DNA
tagtttgtcacccaaccggt

SEQ ID NO: 27 - DNA, 56 bp, synthetic construct
  source 1..56: mol_type = other DNA
acactctttccctacacgacgctcttccgatctagaggtgttgagcagtctctagg

SEQ ID NO: 28 - DNA, 53 bp, synthetic construct
  source 1..53: mol_type = other DNA
cacacctccctaccggatgtctagccttctcgtgtgcagacttgaggtcagtg

SEQ ID NO: 29 - DNA, 19 bp, synthetic construct
  source 1..19: mol_type = other DNA
gcccagacccagcatcatt

SEQ ID NO: 30 - DNA, 21 bp, synthetic construct
  source 1..21: mol_type = other DNA
gggagtgcccaaagctctaaa

SEQ ID NO: 31 - DNA, 104 bp, synthetic construct
  source 1..104: mol_type = other DNA
ctggagtcaggaagctggggatggaatggaagctggtgtcccgccatggggaaggaaggcaccccgaaggctctgagctggagccaggtgggaaccaggggccg

(19) United States
(12) Patent Application Publication
(10) Pub. No.: US 2025/0084442 A1
(43) Pub. Date: Mar. 13, 2025

(54) METHODS OF BIALLELIC MODIFICATION

(71) Applicant: Virginia Tech Intellectual Properties, Inc., Blacksburg, VA (US)

(72) Inventor: Fernando Biase, Blacksburg, VA (US)

(21) Appl. No.: 18/885,348

(22) Filed: Sep. 13, 2024

Related U.S. Application Data
(60) Provisional application No. 63/538,181, filed on Sep. 13, 2023.

(51) Int. Cl. C12N 15/90; C12N 9/22; C12N 15/11

(52) U.S. Cl. CPC C12N 15/907; C12N 9/22; C12N 15/11; C12N 2310/20

(57) ABSTRACT

Described herein are methods and techniques for biallelic modification of DNA.

### CROSS-REFERENCE TO RELATED APPLICATIONS

This application claims the benefit of and priority to U.S. Provisional Patent Application No. 63/538,181, filed on Sep. 13, 2023, the contents of which is incorporated by reference herein in its entirety.

### STATEMENT REGARDING FEDERALLY SPONSORED RESEARCH

This invention was made with government support under Grant No. 2018-67015-31936 awarded by the United States Department of Agriculture National Institute of Food and Agriculture. The government has certain rights in the invention.

### SEQUENCE LISTING

This application contains a sequence listing filed in electronic form as an xml file entitled “VTIP-0450US_ST26.xml”, created on Sep. 12, 2024, and having a size of 28,610 bytes. The content of the sequence listing is incorporated herein in its entirety.

### TECHNICAL FIELD

The subject matter disclosed herein is generally directed to biallelic modification of DNA.

### BACKGROUND

CRISPR-Cas ribonucleoproteins are important tools for gene editing in pre-implantation embryos. However, the inefficient production of biallelic deletions in cattle zygotes has hindered mechanistic studies of gene function. As such improved compositions, methods, and techniques are needed for improved biallelic modifications of DNA in non-human animal zygotes and other contexts.

Citation or identification of any document in this application is not an admission that such a document is available as prior art to the present invention.

### SUMMARY

Described in certain example embodiments herein are methods of biallelic DNA modification including modifying a target DNA using a DNA modification agent; and knocking down target RNA using an RNA modification agent.

In some embodiments, the DNA modification agent is a CRISPR-Cas system, a TALEN, a Zinc Finger Nuclease, an Omega System, a transposase, a recombinase, a recombinase-based system, a non-LTR retrotransposon, or a meganuclease.

In some embodiments, the DNA modification agent is a Type II CRISPR-Cas system. In some embodiments, the DNA modification agent is a CRISPR-Cas9 system. In some embodiments, the DNA modification agent is a CRISPR-Cas9D1-A system.

In some embodiments, the RNA modification agent is a CRISPR-Cas system or RNAi. In some embodiments, the RNA modification agent is a Type VI CRISPR-Cas system. In some embodiments, the RNA modification agent is a CRISPR-Cas13 system. In some embodiments, the RNA modification agent is a CRISPR-Cas13a system.

In some embodiments, modifying, knocking down, or both occurs in vivo or in vitro. In some embodiments, modifying, knocking down, or both occurs in a cell.

In some embodiments, the cell is a zygote. In some embodiments, the cell is a non-human animal cell. In some embodiments, the non-human animal is a bovine, porcine, ovine, canine, feline, or equine.

In some embodiments, delivery of the DNA modifying agent and/or RNA modifying agent occurs via electroporation.

Described in certain example embodiments herein is a modified DNA produced by a method of biallelic modification of the present description.

Described in certain example embodiments herein is a cell including a modified DNA produced by a method of biallelic modification of the present description. In some embodiments, the cell is a zygote. In some embodiments, the cell is a non-human animal cell. In some embodiments, the non-human animal is a bovine, porcine, ovine, canine, feline, or equine.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Targeted DNA deletions using CRISPR-Cas9. Representative images of the DNA mapping of sequences resultant from high-throughput sequencing of embryos electroporated with either Cas9 or Cas9D10A aligned to the cattle genome.

FIG. 2A-2C—Representative schematics of DNA mapping from fully edited blastocysts produced by two sessions of electroporation with Cas9D10A and OCT4-targeting sgRNAs. (FIG. 2A) Genome annotation identifying sgRNA targets and sequencing primers. (FIG. 2B (SEQ ID NO: 31)) Sanger and (FIG. 2C) Nanopore targeted sequencing.

FIG. 3—Knockdown activity of Cas13a in cleavage cattle embryos. Scale bar: 100 μm.

FIG. 4—Schematic of CRISPR-DART procedure. Created with BioRender.com.

FIG. 5A-5E—Impact of OCT4 knockout in cattle pre-implantation embryos. (FIG. 5A) Immunofluorescence assay of OCT4 and NANOG in cattle pre-implantation embryos. Scale bar: 100 μm. (FIG. 5B) In vitro produced blastocysts 188-190 hpf Images are presented in two focal planes for the visualization of the inner cell mass and blastocoel cavity. Scale bar: 100 μm. (FIG. 5C) Schematics of the DNA sequence mapping from three of five blastocysts used for RNA-sequencing. (FIG. 5D) Heatmap depicting the relative differential transcript abundance of 125 genes in OCT4 knockout blastocysts. (FIG. 5E) Transcript abundance of 12 genes functionally associated with the maintenance of pluripotency.

FIG. 6A-6B—CRISPR-Cas in cattle zygotes. (FIG. 6A) Putative zygotes electroporated with Cas9-RFP. (FIG. 6B) In vitro cleavage assay showing the targeted cleavage of DNA by CRISPR-Cas9 and specific sgRNAs. 1: targeted DNA fragment treated with Cas9+sgRNA1, 2: uncut, original DNA fragment; 3: targeted DNA fragment treated with Cas9+sgRNA2.

FIG. 7A-7E—Amplicons produced from embryos electroporated for the deletion of a segment of OCT4. (FIG. 7A) PCR assays of the targeted OCT4 DNA fragment of individual blastocysts electroporated once with either Cas9 or Cas9D10A and sgRNAs. (FIG. 7B) PCR assays of the targeted OCT4 DNA fragment of individual blastocysts electroporated twice with Cas9D10A and sgRNAs. (FIG. 7C) PCR assays of the targeted OCT4 DNA fragment of individual blastocysts electroporated twice with Cas9D10A and sgRNAs. (FIG. 7D) PCR assays of a non-targeted DNA fragment of individual blastocysts electroporated once with either Cas9 or Cas9D10A and sgRNAs. (FIG. 7E) PCR assays of a non-targeted DNA fragment of individual blastocysts electroporated twice with Cas9D10A and sgRNAs.

FIG. 8—Example of alignment with nanopore long sequences (gray) showing the gapped alignment (blue, as represented in greyscale). This embryo was a mosaic with at least one wild-type chromosome sequence. Whole gray arrow point to one example of wild type sequence that covers the entire region of the chromosome targeted by the ribonucleoproteins. Empty arrows point to sequences that were produced from edited chromosomes by the ribonucleoproteins.

FIG. 9—Two examples (Samples 1 and 18) of alignment with nanopore long sequences (gray) showing the gapped alignment (blue, as represented in greyscale), including some minor variants of the deletions.

FIG. 10A-10C—Patterns of RNA-sequencing data aligned to OCT4. (FIG. 10A) Example of two blastocysts lacking exon 1 of the OCT4 gene. Note the absence of transcripts mapping to the introns. (FIG. 10B) Genome browser showing conservation of POU5F1b between human and other species. (FIG. 10C) Example of two wild type blastocysts. Note the presence of transcripts mapping to the introns.

The figures herein are for illustrative purposes only and are not necessarily drawn to scale.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et al., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “control” refers to an alternative subject or sample used in an experiment for comparison purpose and included to minimize or distinguish the effect of variables other than an independent variable.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “culturing” can refer to maintaining cells under conditions in which they can proliferate and avoid senescence as a group of cells. “Culturing” can also include conditions in which the cells also or alternatively differentiate.

As used herein, “deoxyribonucleic acid (DNA)” and “ribonucleic acid (RNA)” can generally refer to any polyribonucleotide or polydeoxyribonucleotide (collectively polynucleotides), which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), long non-coding RNA (lncRNA) ribozymes, aptamers, guide RNA (gRNA), coding mRNA (messenger RNA), cell-free DNA (cfDNA), circulating cfDNA, and/or the like. As used herein, “DNA molecule” can include nucleic acids/polynucleotides that are made of DNA.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides or polynucleotides of the present disclosure, unless stated otherwise.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in aspects modulation may encompass an increase in the value of the measured variable by about 10 to 500 percent or more. In aspects, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In aspects, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In aspects, modulation may be specific or selective, hence, one or more desired phenotypic aspects of a cell or cell population may be modulated without substantially altering other (unintended, undesired) phenotypic aspect(s).

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, a “population” of cells (or “cell population”) is any number of cells greater than 1. In some embodiments, the cell population contains at least 1×10², at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, at least 1×10¹⁰, at least 1×10²⁰, at least 1×10³⁰, at least 1×10⁴⁰, or at least 1×10⁵⁰ cells.

As used herein, “positive control” refers to a “control” that is designed to produce the desired result, provided that all reagents are functioning properly and that the experiment is properly conducted.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “vector” or is used in reference to a vehicle used to introduce an exogenous nucleic acid sequence into a cell. A vector may include a DNA molecule, linear or circular (e.g. plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

As used herein, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The driving force behind gene functionality studies is the targeted alteration of genomic sequences followed by observation of phenotypic deviations. The deletion of functional sequences in the genome, also called knockouts (KO), can be used to study the roles of genes during pre-implantation embryonic development. Mechanistic studies of gene function provide information connecting genome and phenotype during early embryogenesis, and the data may be used to better understand biological function or disease. The CRISPR-Cas system has been the method of choice for most researchers wishing to alter genome sequences in somatic, germ, or embryonic cells. CRISPR-Cas systems have gained traction due to the simplicity of design and synthesis of gRNAs with sequence complementarity to the target region and improved efficiency when compared to other common methods for sequence alterations.

Despite recent advancements in protein engineering giving rise to CRISPR-Cas ribonucleoproteins of greater efficiency and specificity, biallelic deletion efficiency, or the deletion of targeted sequences in both chromosomes, remains low in CRISPR-Cas treated zygotes across many species, including cattle. Interestingly, only four reports provide data on biallelic deletion efficiency in studies utilizing CRISPR-Cas introduced through electroporation of cattle zygotes. These studies averaged 75% of sampled embryos containing partial deletions, with the presence of at least one wildtype allele, and 59% containing full deletions with no wildtype alleles. Some intrinsic factors of zygote biology, such as chromatin compaction and the timing of DNA replication, may impair deletion efficiency due to sequence inaccessibility for CRISPR-Cas binding or the increased number of target sites requiring DNA cleavage. Though the introduction of increased amounts of CRISPR-Cas by more intense electroporation conditions is shown to improve editing efficiencies in cattle zygotes, embryonic mortality increases in tandem. Alternate methods for increasing CRISPR-Cas content in the zygote have been used, such as zona pellucida drilling prior to electroporation in cattle or zona removal in swine. These methods may improve CRISPR-Cas delivery but do not mitigate the setback of embryo mortality. Additionally, it has been suggested that maternally inherited mRNA, present in mammalian zygotes, may support sufficient protein production in the absence of a functional gene. The presence of mRNA resulting from the gene of interest likely hinders gene functionality studies in preimplantation embryos and may be responsible for inconsistent knockout phenotypes. To that end, Cas13a may be used to knockdown maternal or nascent mRNA and further obstruct protein production, but this element has not been accounted for in previous cattle studies. Altogether, many factors can influence the efficiency of CRISPR-Cas systems in pre-implantation embryos.

The gene OCT4, or octamer transcription factor 4, is thought to maintain pluripotency in early cattle and human embryos through its role as a transcription factor for many pluripotency related genes. Additionally, it functions in the HIPPO signaling pathway and is thought to be a key regulator of the first cell lineage differentiation event in cattle. The function of OCT4 has been studied in murine preimplantation embryogenesis models, and these studies show that normal blastocyst development and first cell lineage differentiation are possible in the absence of an OCT4 gene, but one murine model results in the development of blastocysts with absent inner cell mass. As HIPPO signaling processes vary between bovine and murine preimplantation development, these results may not provide adequate translation of information regarding human cell lineage differentiation. Studies to determine the role of OCT4 have been completed by CRISPR-Cas mediated KOs in cattle zygotes, but these studies produced varying outcomes and inconsistent phenotypes. Most studies report OCT4 KO cattle embryos maintaining the ability to reach the blastocyst stage and effectively completing the first cell lineage differentiation event in the absence of this gene. Conversely, one report showed developmental arrest at the morula stage, prior to cell lineage specification. This variability may be due to unaccounted factors, such as maternal or pre-existing mRNA, the common presence of wildtype alleles in CRISPR-Cas genome edited cattle zygotes, and how zygotes were generated. As such improved compositions, methods, and techniques are needed for improved biallelic modifications of DNA in non-human animal zygotes and other contexts.

With that said, embodiments disclosed herein can provide are methods of biallelic modification comprising modification of DNA and knockdown of target RNA in the same environment. In some embodiments the environment is a cell. In some embodiments, the environment is a zygote. In some embodiments, the zygote is a non-human animal zygote. In some embodiments, the non-human animal zygote is a bovine zygote. In some embodiments, the environment is an in vitro environment. In some embodiments, the environment is ex vivo.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Methods of Biallelic DNA Modification

Described in certain example embodiments, the method of biallelic DNA modification includes modifying a target DNA using a DNA modification agent; and knocking down a target RNA using an RNA modification agent. In some embodiments, wherein modifying, knocking down, or both occurs in vivo or in vitro. In some embodiments, wherein modifying, knocking down, or both occurs in a cell. In some embodiments, the cell is a zygote. In some embodiments, the cell is a non-human animal cell. In some embodiments, the non-human animal is a cell, porcine, ovine, canine, feline, or equine cell. In some embodiments, the non-human animal cell is a mouse, rat, or guinea pig cell.

In some embodiments, the target DNA is genomic DNA. In some embodiments, the target RNA is nascent RNA, such as nascent mRNA. In some embodiments, the target RNA is maternally inherited RNA, such as maternally inherited mRNA.

In some embodiments, one or more DNA modification agents are used to target one or more target DNA molecules. In some embodiments, the two or more target DNA molecules are within the same gene. In some embodiments, the two or more target DNA molecules are not within the same gene. In some embodiments, one the two or more target DNA molecules is in a non-coding region of a gene or regulatory region of the gene and one of the two or more target DNA molecules is in a coding region of the same gene.

In some embodiments, the DNA modification agent(s) and/or RNA modification agent(s) are delivered together or separately. In some embodiments, the DNA modification agent(s) and/or RNA modification agent(s) are delivered together or separately in one or more rounds of electroporation. Additional delivery methods are described elsewhere herein.

Without being bound by theory, surprisingly, embodiments of the method of biallelic modification described herein can increase the efficiency of biallelic polynucleotide modifications, such as in a mammalian cell. Using bovine as an exemplary model organism, Applicant observed that the methods described herein can increase genomic modification efficiency in cells with nascent or maternal RNA that hinders genomic modification.

In some embodiments, efficiency of biallelic modification is increased 0.1 to 100-fold or more (e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to/or 100 fold or more) as compared to a suitable control. In some embodiments, the suitable control is a method of modification that does not include knockdown or ablation of nascent or maternal mRNA.

### DNA and RNA Modification Agents

The method can include using a DNA modification agent capable of targeting and/or modifying a target DNA. In some embodiments, the DNA modification agent is a CRISPR-Cas system, a TALEN, a Zinc Finger Nuclease, an Omega System, a transposase, a recombinase, a recombinase-based system, a non-LTR retrotransposon, or a meganuclease. In some embodiments, the DNA modification agent is a Type II CRISPR-Cas system. In some embodiments, the DNA modification agent is a CRISPR-Cas9 system. In some embodiments, the DNA modification agent is a CRISPR-Cas9D1-A system.

In some embodiments, the RNA modification agent is a CRISPR-Cas system or RNAi. In some embodiments, the RNA modification agent is a Type VI CRISPR-Cas system. In some embodiments, the RNA modification agent is a CRISPR-Cas13 system. In some embodiments, the RNA modification agent is a CRISPR-Cas13a system.

Additional exemplary DNA and RNA modification agents are now described in greater detail.

### CRISPR-Cas Systems

In some embodiments, the DNA or RNA modification agents are CRISPR-Cas molecules or a CRISPR-Cas system (also referred to herein as a CRISPR system). In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

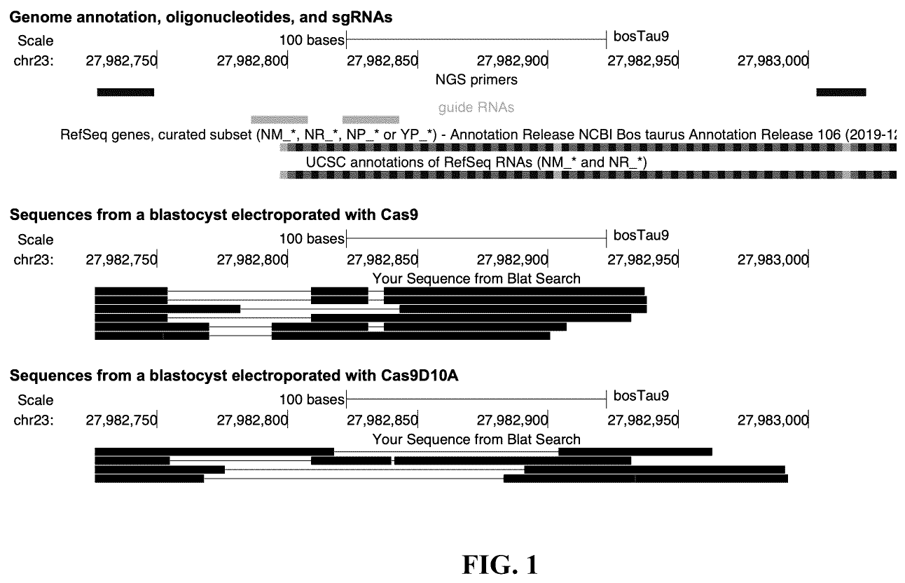

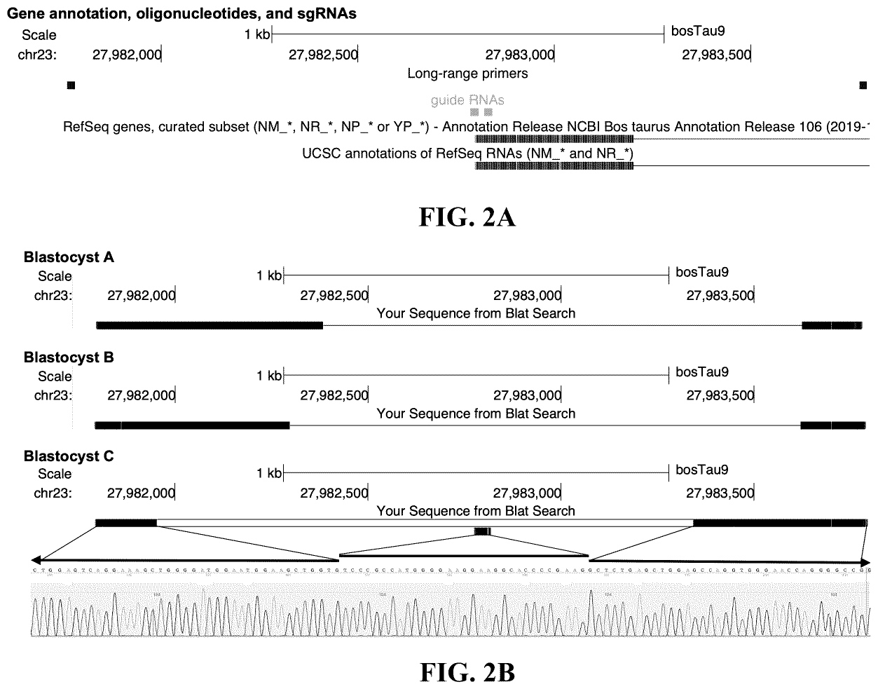

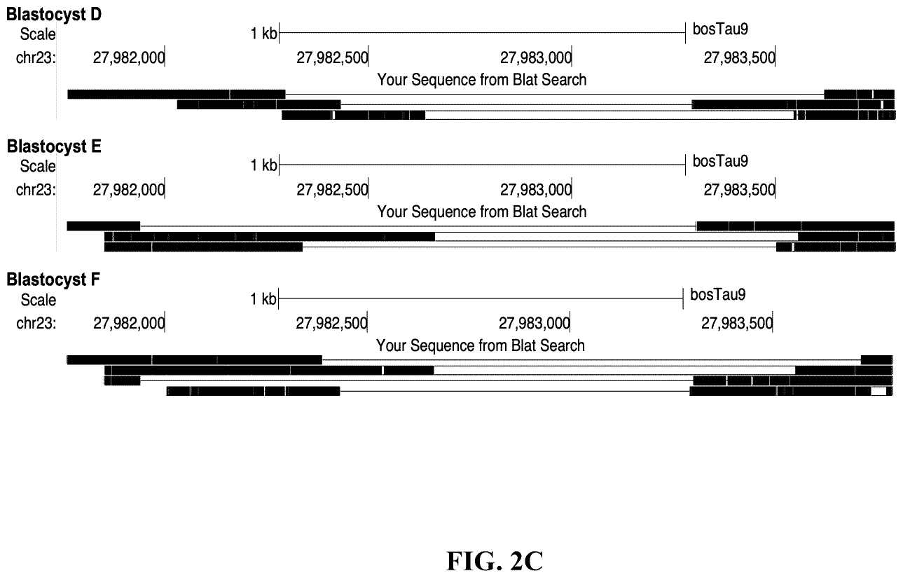

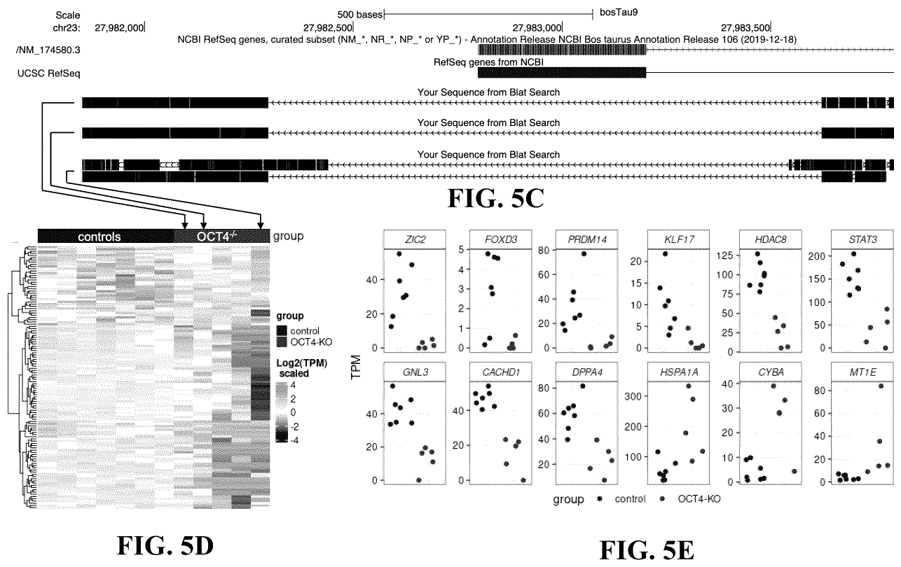

In some embodiments, the CRISPR-Cas system is a Class 1 system, which contains Class 1 Cas proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

In some embodiments, the CRISPR-Cas system is a Class 2 CRISPR-Cas system, which contains Class 2 Cas protein(s). Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at FIG. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

The distinguishing feature of these types of CRISPR-Cas systems is that their effector complexes are composed of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also can display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9. In some embodiments the Cas9 is Cas9D10A.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, Cas13d, Cas13e, Cas13h, Cas13i, Cas13f, Cas13Bt-B, Cas13g, or Cas13Bt-A. See e.g., Hu et al., Cell Discovery volume 8, Article number: 107 (2022).

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sea sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See, e.g., Table A (see also e.g., Gleditzsch et al., RNA Biol. 2019, 16(4), 504-517, Xie et al., Biotechnol. J. 2018, 13(4), e1700561, Lee et al., Mol. Ther. 2016, 24(3), 645-654; Kim et al., Nat. Commun. 2017, 8, 14500; and Muller et al., Mol. Ther. 2016, 24(3), 636-644), which shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 62(1):137-147), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components of the CRISPR-as system can have one or more sequences related to nucleus targeting and transportation. Such sequence may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mx1 protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for indel activity) at the target sequence, or assay for altered gene expression activity affected by DNA-targeting complex formation and/or DNA-targeting), as compared to a control not exposed to the CRISPR-Cas protein, or exposed to a CRISPR-Cas protein lacking the one or more NLSs.

The CRISPR-Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In some embodiments, a component (e.g., a Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Donor Templates

In some embodiments, the CRISPR-Cas system can include a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, or 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, the DNA or RNA modification agent is a DNA or RNA Base Editor or Base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, the DNA or RNA modification agent is a prime editing system. See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

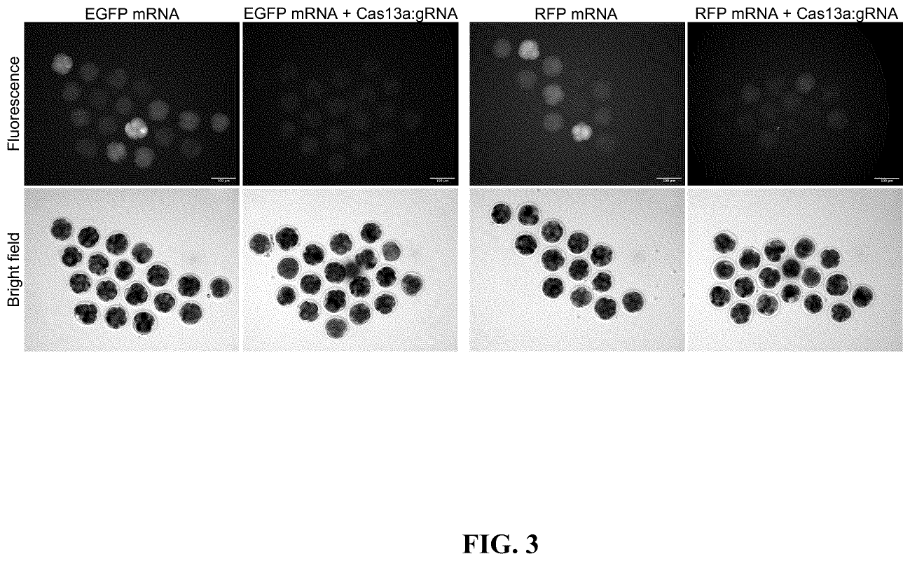

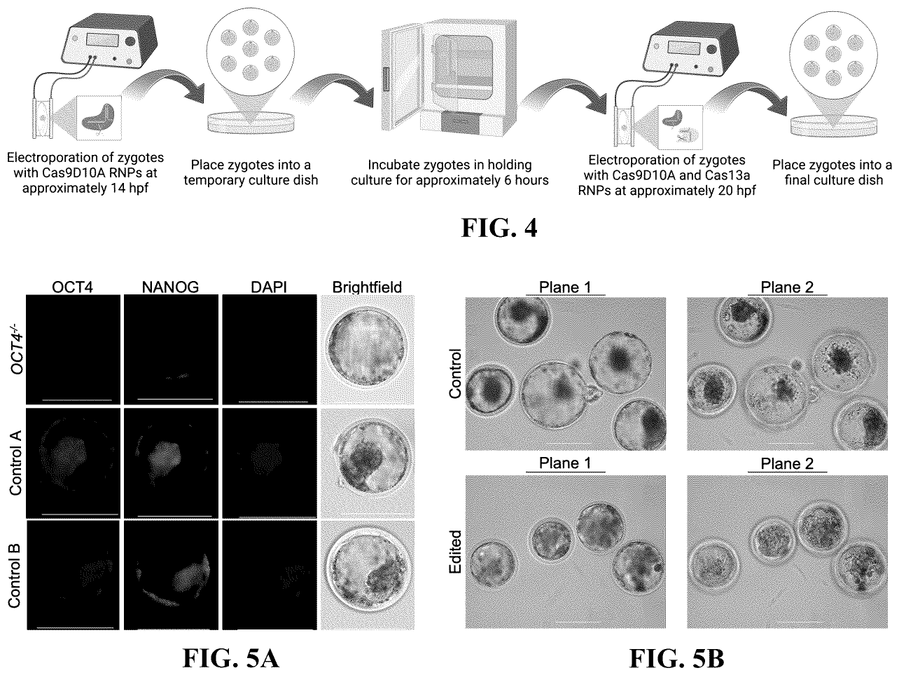

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, the DNA or RNA modification agent is a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### OMEGA Systems

In one example embodiment, the DNA or RNA modification agent is a transposon-encoded RNA-guided nuclease system, referred to herein as OMEGA (Obligate Mobile Element Guided Activity) systems or complexes, or Ω systems or complexes for short. See, e.g., Altae-Tran H, Kannan S, Demircioglu F E, et al. The widespread IS200/IS605 transposon family encodes diverse programmable RNA-guided endonucleases. Science. 2021; 374(6563):57-65. OMEGA systems include, but are not limited to IscB, IsrB, IshB, and TnpB systems.

In some embodiments, the nucleic acid-guided nucleases of an OMEGA system described herein may be an IscB protein (see, e.g., International patent application publication No. WO2022087494A1; and Altae-Tran H, et al. 2021). An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated. In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the nucleic acid-guided nucleases of an OMEGA system herein may be an IsrB (Insertion sequence RuvC-like OrfB) protein (see, e.g., International patent application publication No. WO2022087494A1; and Altae-Tran H, et al. 2021). IsrB refers to a group of shorter, ˜350 aa IscB homologs that are also encoded in IS200/605 superfamily transposons. These proteins contain a PLMP domain and split RuvC but lack the HNH domain.

In some embodiments, the nucleic acid-guided nucleases herein may be a TnpB protein (see, e.g., International patent application publication No. WO2022159892A1; and Altae-Tran H, et al. 2021). TnpB is a putative endonuclease distantly related to IscB and thought to be the ancestor of Cas12, the type V CRISPR effector. The TnpB system comprises a TnpB polypeptide and a nucleic acid component capable of forming a complex with the TnpB polypeptide and directing the complex to a target polynucleotide. TnpB systems are a distinct type of Ω system. The nucleic acid component of Ω systems is structurally distinct from other RNA-guided nucleases, such as CRISPR-Cas systems, and may also be referred to as a ωRNA. In certain example embodiments, the TnpB systems are RNA-predominate, that is the nucleic acid component makes a larger contribution to the overall size of the TnpB complex relative to other RNA-guided nuclease systems such as CRISPR-Cas. Also, given the more minimal structural features of TnpB relative other known programmable nucleases such as CRISPR-Cas, the polynucleotide binding pocket is open and more accessible, which can facilitate greater access to and ability to manipulate, modify, edit, remove, or delete nucleotides at a target region on the bound polynucleotide.

Accordingly, it is contemplated within the scope of the present invention that OMEGA systems may be used in place of CRISPR-Cas systems due to their reprogrammable nature. These embodiments include further modified versions of CRISPR-Cas systems such as base editing systems, prime editing systems, CAST systems, and non-LTR retrotransposons, as discussed elsewhere herein.

### TALE Nucleases

In some embodiments, the DNA modification agent is a TALE nuclease or TALE nuclease system. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. The nucleic acid to which the TALE binds can be DNA. As used herein, the term “polypeptide monomers”, “TALE monomers” or “monomers” is used herein to refer to the highly conserved repetitive polypeptide sequences within the TALE nucleic acid binding domain and the term “repeat variable di-residues” or “RVD” is used herein to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is contained within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD can be alternatively represented as X*, where X represents X₁₂ and (*) indicates that X₁₃ is absent. The DNA binding domain can contain several repeats of TALE monomers and this may be represented as (X₁₋₁₁-(X₁₂X₁₃)-X₁₄₋₃₃ or 34 or 35)_{z}, where in some embodiments, z is at least 5 to 40. In some embodiments, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The TALE polypeptides can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the TALE polypeptides will bind. As used herein the monomers and at least one or more monomers are “specifically ordered to target” the genomic locus or gene of interest. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
MDPIRSRTPSPARELLSGPQPDGVQPTADRGVSPPAGGP
LDGLPARRTMSRTRLPSPPAPSPAFSADSFSDLLRQFDPSLENTS
LFDSLPPFGAHHTEAATGEWDEVQSGLRAADAPPPTMRVAVTA
ARPPRAKPAPRRRAAQPSDASPAAQVDLRTLGYSQQQQEKIKP
KVRSTVAQHHEALVGHGFTHAHIVALSQHPAALGTVAVKYQD
MIAALPEATHEAIVGVGKQWSGARALEALLTVAGELRGPPLQL
DTGQLLKIAKRGGVTAVEAVHAWRNALTGAPLN

An exemplary amino acid sequence of a C-terminal capping region is:

(SEQ ID NO: 19)
RPALESIVAQLSRPDPALAALTNDHLVALACLGGRPAL
DAVKKGLPHAPALIKRTNRRIPERTSHRVADHAQVVRVLGFFQ
CHSHPAQAFDDAMTQFGMSRHGLLQLFRRVGVTELEARSGTLP
PASQRWDRILQASGMKRAKPSPTSTQTPDQASLHAFADSLERD
LDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides described herein.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

### Zinc Finger Nucleases

In some embodiments, the DNA modifying agent is a Zinc Finger Nuclease (ZFN). Zinc Finger proteins can include a functional domain. ZFNs are generally known and were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary ZFNs and general uses can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference and can be adapted for use with the present embodiments described herein.

### Meganucleases

In some embodiments the DNA modifying agent is a meganuclease or system thereof. Meganucleases are generally known and are endodeoxyribonucleases characterized by a large recognition site (double-stranded DNA sequences of 12 to 40 base pairs). Exemplary meganucelases and their general use can be found in U.S. Pat. Nos. 8,163,514, 8,133,697, 8,021,867, 8,119,361, 8,119,381, 8,124,369, and 8,129,134, which are specifically incorporated herein by reference and can be adapted for use with the present embodiments described herein.

### RNAi

In certain embodiments, the DNA or RNA modifying agent is an RNAi molecule(s) (e.g., shRNA, siRNA, piRNA, miRNA, and the like). RNAi molecules are generally known and can reduce or “knock down” the amount of, translation of, or activity of a polynucleotide (e.g., an RNA) to which it binds. See e.g., Traber and Yu, J. Pharmacol. Exp. Ther. 2023 January; 384(1): 133-154; Corydon et al., Mol Ther Nucleic Acids. 2023 Jul. 18:33:469-482; and Haiyong, H. Methods Mol Biol. 2018; 1706: 293-302). In some embodiments, the RNAi can reduce the amount of, translation of, or activity of a target polynucleotide (e.g. a target RNA) by at least about 5%, about 10%, about 20%, about 30%, about 40%, about 50%, about 60%, about 70%, about 80%, about 90%, about 95%, about 99%, about 100% of the original RNA level. In some embodiments, the target RNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Non-LTR Retrotransposon Systems

In some embodiments the DNA or RNA modification agent is a Non-LTR Retrotransposon system.

The Non-LTR retrotransposon system can include one or more components of a retrotransposon, e.g., a non-LTR retrotransposon. Native or wild-type non-LTR retrotransposons encode the protein machinery necessary for their self-mobilization. The non-LTR retrotransposon element comprises a DNA element integrated into a host genome. The DNA element may encode one or two open reading frames (ORFs). For example, the R2 element of Bombyx mori encodes a single ORF containing reverse transcriptase (RT) activity and a restriction enzyme-like (REL) domain. Li elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon can include transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. In an example embodiment, the RNA-transposase complex nicks the genome and the 3′ end of the nicked DNA serves as a primer to allow the reverse transcription of the transposon RNA into cDNA. The transposase proteins may then integrate the cDNA into the genome.

Elements of these systems may be engineered to work within the context of the invention. For example, a non-LTR retrotransposon polypeptide may be fused to a programmable nuclease or polynucleotide guided nuclease (e.g., a Cas protein, Omega protein, etc.). The binding elements that allow a non-LTR retrotransposon polypeptide to bind to the native retrotransposon DNA element, can be engineered into a donor construct to facilitate entry of a donor polynucleotide sequence into a target polypeptide.

In certain embodiments, the protein component of the non-LTR retrotransposon may be connected to or otherwise engineered to form a complex with a programmable nuclease, e.g., a Cas polypeptide and the like. The retrotransposon RNA may be engineered to encode a donor polynucleotide sequence. Thus, in certain example embodiments, the Cas polypeptide, via formation of a CRISPR-Cas complex with a guide sequence, directs the retrotransposon complex (i.e., the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence. Example non-LTR retrotransposon systems are disclosed in WO 2021/102042, WO 2022/173830, which are incorporated herein by reference.

Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805, which are incorporated by reference herein in their entireties.

Examples of the non-LTR retrotransposon polypeptides also include R2 from Clonorchis sinensis, or Zonotrichia albicollis. Example non-LTR retrotransposon polypeptides and binding components (5′ and 3′ UTRs) that may be used in the context of the invention are listed in Table 1 along with codon optimized variants of the non-LTR retrotransposons for expression in eukaryotic cells.

A non-LTR retrotransposon may comprise multiple retrotransposon polypeptides or polynucleotides encoding same. In some embodiments, the retrotransposon polypeptides may form a complex. For example, a non-LTR retrotransposon is a dimer, e.g., comprising two retrotransposon polypeptides forming a dimer. The dimer subunits may be connected or form a tandem fusion. A Cas protein or polypeptide may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a Cas protein or polypeptide.

The retrotransposon polypeptides may be enzymes or variants thereof. In some examples, a retrotransposon polypeptide may be a reverse transcriptase, a nuclease, a nickase, a transposase, nucleic acid polymerase, ligase, or a combination thereof. In one example, a retrotransposon polypeptide is a reverse transcriptase. In another example, a retrotransposon polypeptide is a nuclease. In another example, a retrotransposon polypeptide is nickase. In a particular example, a non-LTR retrotransposon comprises a first retrotransposon polypeptide and a second retrotransposon polypeptide, wherein the second retrotransposon polypeptide comprises nuclease or nickase activity. In certain cases, a retrotransposon polypeptide may comprise an inactive enzyme. For example, a retrotransposon polypeptide may comprise a nuclease domain that is inactivated. Such inactivated domain may serve as a nucleic acid binding domain.

The retrotransposon polypeptides may comprise one or more modifications to, for example, enhance specificity or efficiency of donor polynucleotide recognition, target-primed template recognition (TPTR), and/or reduce or eliminate homing function. The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol12 promoter, a pol13 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In some embodiments, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end, the 3′ end, or a location in between.

In some embodiments, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Recombinase-Based Modification Agents

Prime editing and twinPE systems can also be further combined with site-specific recombinases, such as integrases, to facilitate even larger insertions, substitutions and deletions. See e.g., WO 2021/138469; Anzalone A V, Gao X D, Podracky C J, et al. Programmable deletion, replacement, integration and inversion of large DNA sequences with twin prime editing. Nat Biotechnol. 2022; 40(5):731-740; Yarnall et al., Nat Biotechnol (2022). doi.org/10.1038/s41587-022-01527-4, which is incorporated by reference as if expressed in its entirety herein. The prime editing system is used to insert a recombinase recognition site at the desire site of modification and an integrase facilitates the insertion of a donor sequence from a donor template. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

In example embodiments, the recombinase of the present invention is a serine integrase. In example embodiments, serine integrases specifically recombine when recognizing the two attachment sites specific for the integrase. In example embodiments, the heterologous sites are referred to as attP and attB, however, these terms refer to the specific sequences recognized by the specific integrase and do not refer to a single consensus sequence. Serine integrases mediate site-specific recombination between short recognition sites located in phage genomes and bacterial chromosomes, respectively, the attachment site of phage (attP) and attachment site of bacteria (attB) (i.e., the target sites of the integrase), to form the hybrid attachment sites attL and attR. Unlike Cre and Flp recombinases that catalyze reversible site-specific recombination reactions, serine integrases are unidirectional and catalyze only attP and attB recombination without RDF or Xis accessory proteins. Thus, in the absence of any accessory factors integrase is unidirectional. In addition, DNA substrates identified by serine integrases (attP and attB) are relatively short (30-50 bp) and have a minimal length of approximately 34-40 base pairs (bp) (Groth A C et al., Proc. Natl. Acad. Sci. USA 97, 5995-6000 (2000)). The compatibility of distinct DNA topological structures is also quite different from recognition of DNA by Hin recombinase or Tn3 resolvase. Serine integrases recognize DNA substrates specifically, not at random, but can facilitate recombination at sequences with partial identity with wild-type recombination sites, termed pseudo attachment sites (either pseudo attP or pseudo attB). A “pseudo-recombination site” is a DNA sequence recognized by a recombinase enzyme such that the recognition site differs in one or more base pairs from the wild-type recombinase recognition sequence and/or is present as an endogenous sequence in a genome that differs from the genome where the wild-type recognition sequence for the recombinase resides. “Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site. Specific attB and attP sequences for use in the present invention include all wildtype sequences as well as pseudo attB and ataP sequences.

Recombination sites used in the present methods include those recognized by unidirectional, site-directed recombinases (e.g., integrases). Non-limiting examples of serine integrases and recombination sites applicable to the present invention include ϕC31 integrase, Bxb1, ϕBT1 integrase, A118, TP901-1, and R4 and the corresponding recombination sites for each (see, e.g., Groth, A. C. and Calos, M. P. (2004) J. Mol. Biol. 335, 667-678; Lei, et al., FEBS Lett. 2018 April; 592(8):1389-1399; Singh, et al., Attachment Site Selection and Identity in Bxb1 Serine Integrase-Mediated Site-Specific Recombination, PLoS Genet. 2013 May; 9(5):e1003490; and Gupta, et al., Nucleic Acids Res. 2007 May; 35(10): 3407-3419). Additional serine recombinases and recombination sites may be any of those disclosed in US 20180346934A1 and US 2010/0190178. In certain embodiments, a functional domain of the serine integrase is used.

In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### Transposon/Transposase Systems

In some embodiments, the DNA or RNA modification agent can be a transposon/transposase system. Such systems are generally known in the art. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the retrotransposon includes long terminal repeats. In some embodiments, the retrotransposon does not include long terminal repeats. In some embodiments, the transposon is a DNA transposon system. DNA transposon systems can include a transposase. In some embodiments, the transposon system is configured as a non-autonomous transposon, meaning that the transposition does not occur spontaneously on its own. In some of these embodiments, the transposon system lacks one or more polynucleotide sequences encoding proteins or the proteins required for transposition. In some embodiments, the non-autonomous transposon systems lack one or more Ac elements. The missing components can be spatially and/or temporally provided to complete the system thus triggering activity of the system and transposition and allowing for spatial and/or temporal control of the transposon system activity. In some embodiments, the transposon system can be used for gene trapping.

Any suitable transposon system can be used. Suitable transposon and systems thereof can include, without limitation, Sleeping Beauty transposon system (Tc1/mariner superfamily) (see e.g. Ivics et al. 1997. Cell. 91(4): 501-510), piggyBac (piggyBac superfamily) (see e.g. Li et al. 2013 110(25): E2279-E2287 and Yusa et al. 2011. PNAS. 108(4): 1531-1536), Tol2 (superfamily hAT), Frog Prince (Tc1/mariner superfamily) (see e.g. Miskey et al. 2003 Nucleic Acid Res. 31(23):6873-6881) and variants thereof.

### Delivery of the DNA and RNA Modification Agents

Delivery of the DNA and/or RNA modification agent(s) to a target polynucleotide and/or cell can be by any suitable method. In some embodiments, a physical delivery method is used to deliver the DNA and/or RNA modification agent(s) to a target polynucleotide and/or cell. Physical methods include, without limitation, microinjection, electroporation, hydrodynamic delivery, transfection, transduction, and biolistics. In some embodiments, a vector-based delivery method is used to deliver the DNA and/or RNA modification agent(s) to a target polynucleotide and/or cell. In some embodiments, delivery employs a delivery vehicle. Various delivery methods are described in greater detail in e.g., International Pat. Pub. WO 2024163717A1, specifically at paragraphs [0170]-[0229], which is incorporated by reference as if expressed in its entirety herein.

In some embodiments, delivery of the DNA modification agent and/or RNA modification agent occurs via electroporation. In some embodiments, a first round of electroporation with the DNA modification agent followed by a second round of electroporation with the RNA modification agent. In some embodiments, a first round of electroporation with the RNA modification agent followed by a second round of electroporation with the DNA modification agent. In some embodiments, one or more rounds of electroporation with the DNA modification agent followed by a one or more rounds of electroporation with the RNA modification agent. In some embodiments, the DNA and the RNA modification agents can be delivered via electroporation simultaneously. The working examples herein provide exemplary methods and techniques for performing electroporation. Additional electroporation techniques an be found in e.g., Biase and Schettini. STAR Protocols. 2024, 5, 102940; Alghadban et al., Sci Rep. 2020; 10: 17912; Troder et al., PLoS One. 2018; 13(5): e0196891; Lin and Van Enennaam. Front. Genet. 2021. Vol. 12, doi. 10.3389/fgene.2021.648482; Biase and Schettini. STAR Protocols. 2024, 5, 102940; Tanihara et al., Front. Cell Dev. Biol., 2023, 11, doi.org/10.3389/fcell.2023.884340; Tanihara et al., Int J Mol Sci. 2021 March; 22(5): 2249; Punetha et al., Animals (Basel). 2024 January; 14(1): 134; Mahdi et al., Int J Mol Sci. 2022 Sep. 6; 23(18); Pi et al., Int. J. Mol. Sci. 2024, 25(17), 9145, which can be adapted for use with the present description herein.

### Modified Polynucleotides, Cells, and Organisms

Described herein are modified polynucleotides (e.g., target polynucleotides), cells, cell populations, and organisms that can be generated using a method of biallelic modification of the present embodiments described in greater detail elsewhere herein. The modified target polynucleotides cells, cell populations, and organisms can have an insertion of one or more nucleotides, deletion of one or more nucleotides, mutation of one or more nucleotides, or any combination thereof. The modification can result in activation of one or more genes, inactivation of one or more target polynucleotides (including, but not limited to, genes), modulation of one or more target polynucleotides, or a combination thereof. Cells, including cells in an organism, can be modified in vitro, in situ, ex vivo, or in vivo. In some embodiments, the modification is an insertion, deletion, and/or mutation of one or more nucleotides in a polynucleotide, gene, or allele(s) of interest. In some embodiments, the modification is biallelic. In some embodiments, the modification is in the genome. In other words, in some embodiments, the target polynucleotide(s) are in the genome of a cell. In some embodiments, the modified target polynucleotide is DNA. In some embodiments, the modified target polynucleotide is RNA. In some embodiments, the biallelic modification results in ablation of the polynucleotide of interest.

### Modified Cells

Also described herein are modified cells and cell populations that can be modified by an embodiment of a method of biallelic modification described in greater detail elsewhere herein. In some embodiments, a cell is modified by a DNA and/or RNA modification agent, including but not limited to, a programmable nuclease-based system such as a TALEN, Zinc-finger nuclease, CRISPR-Cas or Cas-based system, or an Omega system; recombinase or recombinase-based system; transposon system, RNAi, and/or the like. Exemplary DNA and RNA modification agents are described in greater detail elsewhere herein. In some embodiments, the cell is a eukaryotic cell. In some embodiments, the eukaryotic cell is a mammalian cell. In some embodiments, the eukaryotic cell is a non-human mammalian cell. In some embodiments, the cell is a human cell. In some embodiments, the modified cell is a zygote.

The cells can be modified in vitro, ex vivo, or in vivo. The cells can be modified by delivering one or more polynucleotide (e.g., DNA RNA) modifying agents or systems described in greater detail elsewhere herein or a component thereof into a cell by a suitable delivery mechanism. Suitable delivery methods and techniques include but are not limited to, transfection via a vector, transduction with viral particles, electroporation, endocytic methods, and others, which are described elsewhere herein and will be appreciated by those of ordinary skill in the art in view of this disclosure. In some embodiments, delivery is via electroporation

The modified cells can be further cultured and/or expanded in vitro or ex vivo using any suitable cell culture techniques or conditions, which unless specified otherwise herein, will be appreciated by one of ordinary skill in the art in view of this disclosure. In some embodiments, the cells can be modified, optionally cultured and/or expanded, and administered to a subject in need thereof. In some embodiments, cells can be isolated from a subject, subsequently modified and optionally cultured and/or expanded, and administered back to the subject. Such administration can be referred to as autologous administration. In some embodiments, cells can be isolated from a first subject, subsequently modified, cultured and/or expanded, and administered to a second subject, where the first subject and the second subject are different. Such administration can be referred to as non-autologous administration or allogeneic.

### Organisms

Also described herein are modified organisms. In some embodiments, the modified organisms can include one or more modified cells as are described elsewhere herein. In some embodiments, the modified organism is a non-human mammal. The modified organisms can be generated using a that can be modified by an embodiment of the method of biallelic modification described herein. Methods of making modified organisms are described in greater detail elsewhere herein.

The systems and methods of the present description can be used to generate modified non-human animal organisms. The system and methods described herein can be used to modify non-germline cells in a human. In some embodiments, the modification is expression of a polynucleotide of interest, gene of interest, and/or allele of interest. In some embodiments, the modification is biallelic. In some embodiments, the modification is an insertion of one or more nucleotides, deletion of one or more nucleotides, mutation of one or more nucleotides, or any combination thereof in a target polynucleotide.

### Non-Human Animals

The systems and methods of the present description may be used to generate modified non-human animals and cells thereof, such as those with biallelic modifications. In some embodiments, the modified organism is a non-human eukaryotic organism; preferably a multicellular eukaryotic organism, comprising one or more modified eukaryotic cells, where the one or more modified cells have been modified according to a method of the present description. In other aspects, the invention provides a eukaryotic organism; preferably a multicellular eukaryotic organism, comprising one or more modified eukaryotic cells according to any of the described embodiments. In some embodiments, the non-human animal is a bovine, porcine, ovine, canine, feline, or equine. In some embodiments, the non-human animal is a mouse, rat, or guinea pig. Methods of generating modified non-human animals have been described and whose techniques can be adapted for use with the embodiments described herein. See e.g., Keefer et al., Proc Natl Acad Sci USA. 2015 Jul. 21; 112(29): 8874-8878; Lee et al., (Proc Natl Acad Sci USA. 2014 May 20; 111(20):7260-5); Lee at al., Arch Pharm Res. 2018; 41(9): 885-897; Tan et al. (Proc Natl Acad Sci USA. 2013 Oct. 8; 110(41): 16526-16531; Mali P, et al. (2013) RNA-Guided Human Genome Engineering via Cas9. Science 339(6121):823-826; Heo et al. (Stem Cells Dev. 2015 Feb. 1; 24(3):393-402; Sid and Schusser et al 2018. Front. Genet. Doi.org/10.3389/fgene.2018.00456) and other avians (e.g. Scott et al. 2010. ILAR J. 51(4):353-361), cattle (Yum et al., 2016. Scientific Reports. 6:27185 and Tait-Burkard et al. 2018. Genome Biology. 19:2014.), sheep and goats (see e.g. Kalds et al., 2019. Front. Genet. Doi.org//10.3389/fgene.2019.00750), horses (see e.g. West and Gill. 2016. J. Equine Vet. Sci. 41:1-6), dogs (see e.g. D. Duan. Nature Biomedical Engineering. 2018. 2: 795-796), reptiles (see e.g. Rasys et al. 2019. Cell Reports. 28:2288-2292), fish (including but not limited to zebrafish, see e.g. Datsomor et al. 2019. Scientific Reports. 9:7533, Liu et al. 2019. Front. Cell. Dev. Biol. https://doi.org/10.3389/fcell.2019.00013), insects (see e.g. Kotwica-Rolinska et al. 2019. Front. Physiol. https://doi.org/10.3389/fphys.2019.00891; Gantz and Akbari. 2018. Curr. Opin. Insect. Sci. 28:66-72), rabbits (see e.g. Kawano and Honda. 2017. Methods Mol. Biol. 4630:109-120; Liu et al., 2018. Nature Commun. 9:2717; and Liu et al. 2018. Gene. https://doi.org/10.1016/j.gene.2018.01.044), mice (see e.g. Hall et al. 2018. Curr Protoc Cell Biol. 81(1): e57), rats (see e.g. Back et al. 2019. Neuron. 102(1):105-119), amphibians (see e.g. Nakayama et al. 2013. Genesis. 51(12):835-843), nematodes (see e.g. J. B. Lok. 2019. Front. Genet. https://doi.org/10.3389/fgene.2019.00656), molluscs (see e.g. Abe and Kuroda. 2019. Development. 146: dev175976 doi: 10.1242/dev.175976, geckos, shrimp and other crustaceans (see e.g. Gui et al. Genes Genomes Genetics: 6(11): 3757-3764), oysters (Yu et al. 2019; March Biotechnol (NY) 21(3):301-309. doi: 10.1007/s10126-019-09885-y), sponges (see e.g. Revilla-i-Domingo et al. 2018. Genetics. 210(2)435-443), dogs (see e.g., Kim et al., BMC Biotechnology volume 22, Article number: 19 (2022)); and cats (see e.g., Yin et al., Biology of Reproduction, Volume 78, Issue 3, 1 Mar. 2008, Pages 425-431; and Lee et al., Scientific Reports volume 14, Article number: 4987 (2024)), the teachings of which can be adapted for use with one or more of the DNA or RNA modification agent(s) and/or systems described herein to generate the modified non-human animal or cell thereof.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Ablation of OCT4 Function in Cattle Embryos by Double Electroporation of CRISPR-Cas for DNA and RNA Targeting (CRISPR-DART)

### Introduction

The driving force behind gene functionality studies is the targeted alteration of genomic sequences followed by observation of phenotypic deviations. The deletion of functional sequences in the genome, also called knockouts (KO), can be used to study the roles of genes during pre-implantation embryonic development (1). Mechanistic studies of gene function provide information connecting genome and phenotype during early embryogenesis, and the data may be used to better understand biological function (2-5) or disease (6-8). The CRISPR-Cas system has been the method of choice for most researchers wishing to alter genome sequences in somatic (9-11), germ (12-15), or embryonic cells (16-25). CRISPR-Cas systems have gained traction due to the simplicity of design and synthesis of gRNAs with sequence complementarity to the target region (26) and improved efficiency when compared to other common methods for sequence alterations (27-30).

Despite recent advancements in protein engineering giving rise to CRISPR-Cas ribonucleoproteins of greater efficiency and specificity (31), biallelic deletion efficiency, or the deletion of targeted sequences in both chromosomes, remains low in CRISPR-Cas treated zygotes across many species, including cattle (32-37). Interestingly, only four reports provide data on biallelic deletion efficiency in studies utilizing CRISPR-Cas introduced through electroporation of cattle zygotes (33, 35, 38, 39). These studies averaged 75% of sampled embryos containing partial deletions, with the presence of at least one wildtype allele, and 59% containing full deletions with no wildtype alleles. Some intrinsic factors of zygote biology, such as chromatin compaction and the timing of DNA replication, may impair deletion efficiency due to sequence inaccessibility for CRISPR-Cas binding or the increased number of target sites requiring DNA cleavage. Though the introduction of increased amounts of CRISPR-Cas by more intense electroporation conditions is shown to improve editing efficiencies in cattle zygotes, embryonic mortality increases in tandem (40). Alternate methods for increasing CRISPR-Cas content in the zygote have been used, such as zona pellucida drilling prior to electroporation in cattle (35) or zona removal in swine (41). These methods may improve CRISPR-Cas delivery but do not mitigate the setback of embryo mortality. Additionally, it has been suggested that maternally inherited mRNA, present in mammalian zygotes (42-45), may support sufficient protein production in the absence of a functional gene. The presence of mRNA resulting from the gene of interest likely hinders gene functionality studies in preimplantation embryos and may be responsible for inconsistent knockout phenotypes. To that end, Cas13a (46) may be used to knockdown maternal or nascent mRNA and further obstruct protein production, but this element has not been accounted for in previous cattle studies. Altogether, many factors can influence the efficiency of CRISPR-Cas systems in pre-implantation embryos.

The gene OCT4, or octamer transcription factor 4, is thought to maintain pluripotency in early cattle (47, 48) and human (49, 50) embryos through its role as a transcription factor for many pluripotency related genes (49, 51). Additionally, it functions in the HIPPO signaling pathway (52) and is thought to be a key regulator of the first cell lineage differentiation event in cattle. The function of OCT4 has been studied in murine preimplantation embryogenesis models, and these studies show that normal blastocyst development and first cell lineage differentiation are possible in the absence of an OCT4 gene (53, 54), but one murine model results in the development of blastocysts with absent inner cell mass (49). As HIPPO signaling processes vary between bovine and murine preimplantation development (52, 55), these results may not provide adequate translation of information regarding human cell lineage differentiation. Studies to determine the role of OCT4 have been completed by CRISPR-Cas mediated KOs in cattle zygotes, but these studies produced varying outcomes and inconsistent phenotypes (35, 36, 56, 57). Most studies report OCT4 KO cattle embryos maintaining the ability to reach the blastocyst stage and effectively completing the first cell lineage differentiation event in the absence of this gene (38, 58, 59). Conversely, one report showed developmental arrest at the morula stage, prior to cell lineage specification (35). This variability may be due to unaccounted factors, such as maternal or pre-existing mRNA, the common presence of wildtype alleles in CRISPR-Cas genome edited cattle zygotes, and how zygotes were generated.

Here, Applicant aimed to improve the efficiency of CRISPR-Cas mediated biallelic deletions in cattle zygotes while degrading preexisting RNAs transcribed from the target gene. Applicant targeted the OCT4 gene, given the inconsistency of results from previous reports. Applicant hypothesized that ribonucleoproteins formed with CRISPR-Cas9D10A produce larger deletions at greater consistency and efficiency than CRISPR-Cas9, and CRISPR-Cas13a can efficiently knockdown mRNA in cattle zygotes. Applicant also hypothesized that simultaneous targeting of DNA and RNA could ablate gene function in cattle zygotes in vitro. In this study, Applicant mitigated the barriers of poor deletion efficiency and the presence of preexisting mRNA while maintaining embryo survival. The dual delivery of CRISPR-Cas9D10A, six-hours apart, increases the incidence of gene editing and full deletions. Additionally, Applicant targeted maternally inherited transcripts with CRISPR-Cas13a while simultaneously removing a targeted sequence of the genome. Altogether, Applicant developed a method for high efficiency genome and transcriptome editing in bovine zygotes using CRISPR-Cas editing technology. Applicant's approach overcomes many limitations of gene editing for mechanistic studies of gene function in pre-implantation embryos. Although cattle blastocyst formation is possible in the absence of OCT4, these embryos lack an inner cell mass and present severe transcriptional dysregulation of several genes related to stemness.

### Results

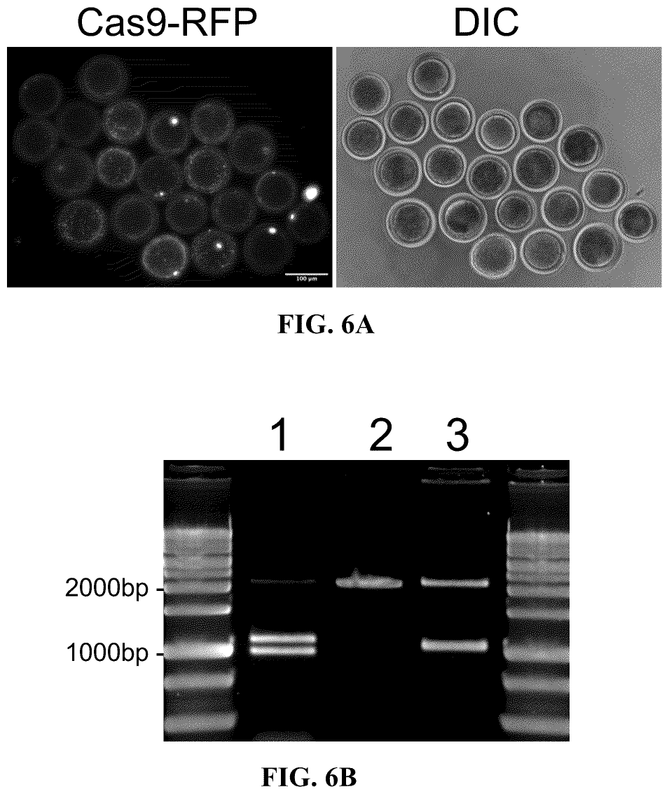

First, Applicant assessed the efficacy of electroporation and the cleavage function of the ribonucleoproteins (RNPs). Here, Applicant used electroporation conditions modified from a previous publication (35), as follows: six poring pulses of 15 volts, with 10% decay, for two milliseconds with a 50-millisecond interval, immediately followed by five transfer pulses of 3 volts, 40% decay, for 50 milliseconds with a 50-millisecond interval, alternating the polarity. Fluorescence imaging showed that the RNP formed by Cas9-RFP+scramble guide RNAs (gRNAs) bypassed the zona pellucida in nearly all putative zygotes (PZ) electroporated (FIG. 6A, Appendix of Nix et al., Proc. Natl. Acad. Sci. U.S.A. Nexus), 2023, 2(11), 1-14)). Next, Applicant confirmed that the RNPs formed with Cas9+OCT4 single guide RNA (sgRNA) 1 or Cas9+OCT4sgRNA2 were able to cleave the targeted DNA in vitro (FIG. 6B, Appendix of Nix et al., Proc. Natl. Acad. Sci. U.S.A. Nexus), 2023, 2(11), 1-14)).

### Both CRISPR-Cas9 and CRISPR-Cas9D10A Produce Deletions in Cattle Zygotes

First, Applicant asked if Cas9 and Cas9D10A would result in similar editing efficiencies and deletion patterns. High-throughput targeted sequencing revealed that 73.1% and 81.5% of embryos presented at least one segment of DNA deleted when Applicant used Cas9 (N embryos=26) or Cas9D10A (N embryos=27), respectively. Applicant observed that 15.4% and 25.9% of the embryos electroporated with Cas9 or Cas9D10A, respectively, and genotyped by sequencing, did not have a wild-type copy of the DNA in the targeted region. The deletions resultant from Cas9 or Cas9D10A varied in their location and length. Applicant observed that Cas9D10A RNPs produced longer deletions and removed the segment of DNA that included both sgRNAs, whereas Cas9 mostly produced small deletions in the region surrounding the sgRNAs but did not cause many deletions spanning both sgRNAs (FIG. 1).

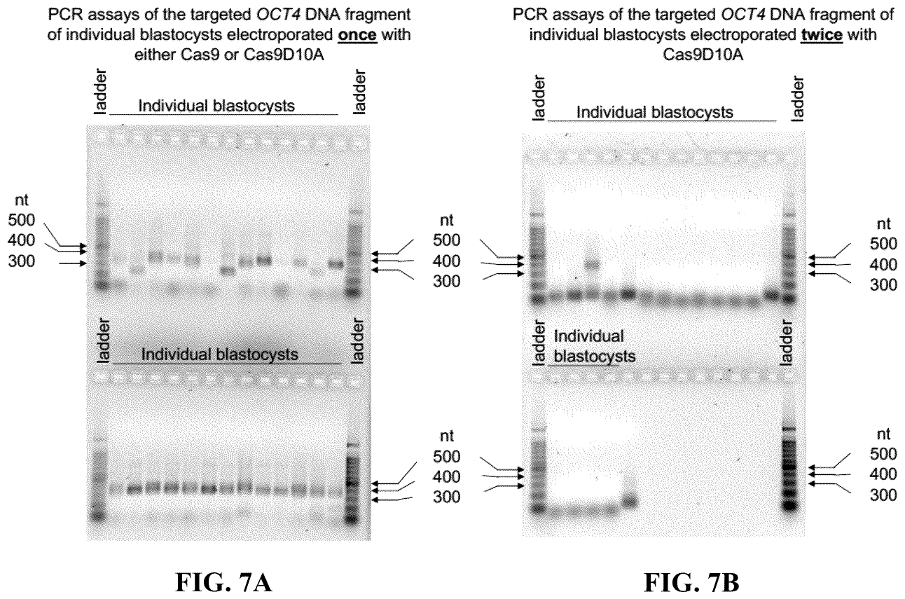

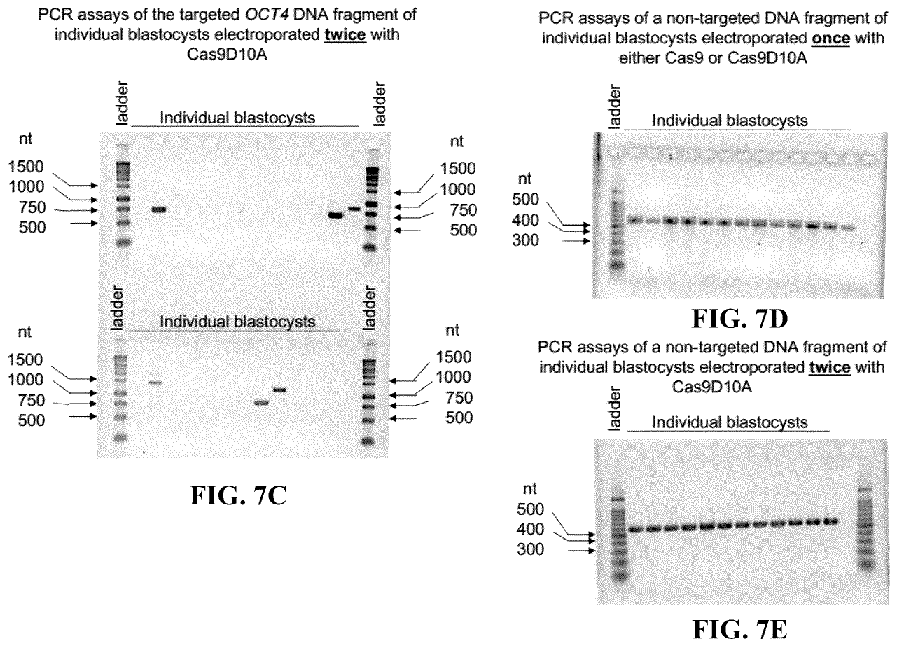

Although not significant (P=0.27, Fisher's Exact test), Cas9D10A produced 10.5 percentage points more full deletions, with no wildtype alleles, when compared to Cas9. Thus, Applicant carried out the next experiments with Cas9D10A and OCT4-targeting sgRNAs. Also, considering that many of the Cas9-RFP RNPs remained in the membrane or perivitelline space (FIG. 6A, Appendix to Nix et al., Proc. Natl. Acad. Sci. U.S.A. Nexus, 2023, 2(11), 1-14), Applicant reasoned that a second electroporation would increase the efficiency of full deletions in cattle presumptive zygotes (PZ). A second electroporation of PZ (approximately six hours after the first electroporation; see methods for details) with RNPs composed of Cas9D10A and associated sgRNAs resulted in no PCR amplification for most blastocysts when using the oligonucleotides designed for high-throughput short-read sequencing (FIG. 7A-7B, Appendix of Nix et al., Proc. Natl. Acad. Sci. U.S.A. Nexus, 2023, 2(11), 1-14). This outcome, and prior reports that CRISPR-Cas9 can produce unexpected large deletions (60, 61), prompted us to design oligonucleotides to flank a wider region of the DNA surrounding Applicant's sgRNA target sequences. Approximately 19% of the blastocysts tested with this long-range pair of oligonucleotides produced an amplicon (FIG. 7C, Appendix of Nix et al., Proc. Natl. Acad. Sci. U.S.A. Nexus, 2023, 2(11), 1-14). All blastocysts that had no amplicon produced with oligos surrounding Applicant's targeting sgRNAs were tested for amplification of a non-targeted autosomal region of the genome to confirm that an embryo was present in the tube (FIG. 7D-7E, Appendix of Nix et al., Proc. Natl. Acad. Sci. U.S.A. Nexus, 2023, 2(11), 1-14; see Supplemental Text, Methods herein, and Appendix of Nix et al., Proc. Natl. Acad. Sci. U.S.A. Nexus, 2023, 2(11), 1-14.

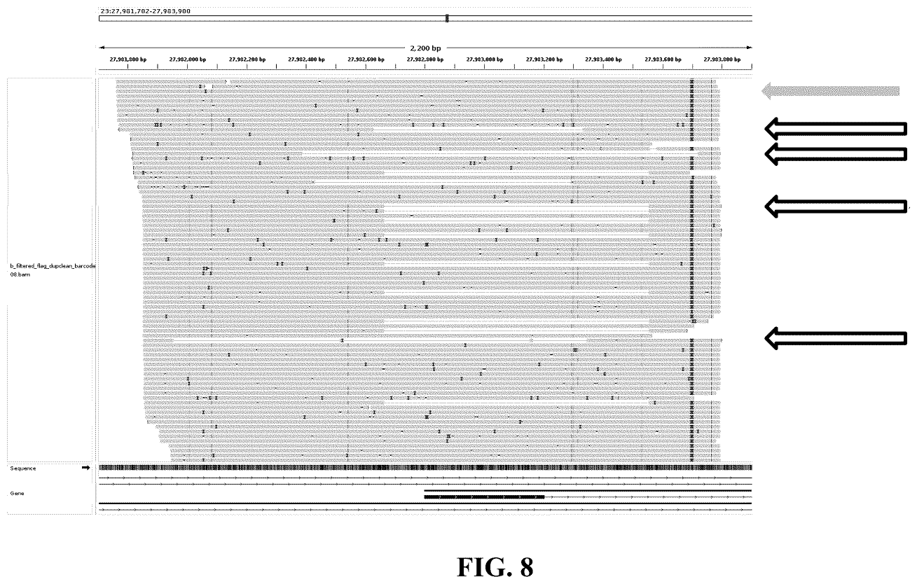

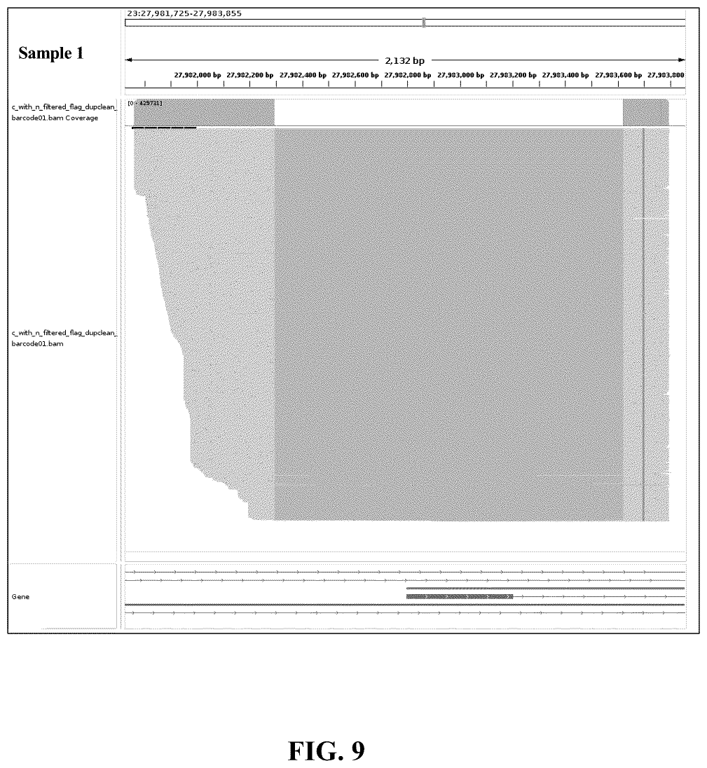

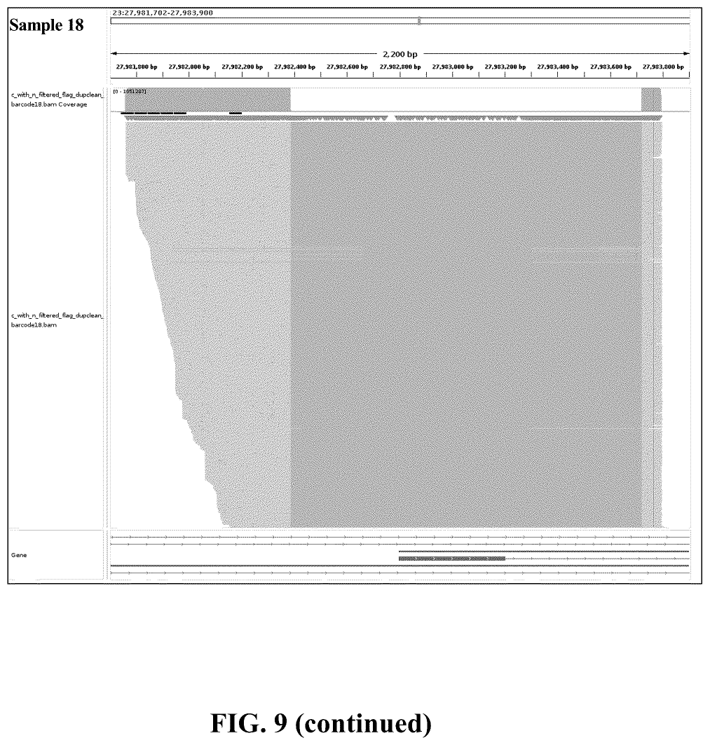

Applicant sequenced the PCR products from seven blastocysts using the Sanger procedure, and three of these samples produced electropherograms from only one fragment (FIG. 2A). The long-range PCR produced multiple amplicons in the other three samples, which is unsuitable for Sanger sequencing. Therefore, Applicant decided to proceed with Nanopore sequencing for multiple allele detection. Twenty-four blastocysts produced amplicons with long-range oligos and were sequenced by Sanger (FIG. 2A) or Nanopore (FIG. 2B) methods. Sequencing results showed that 95.8% (23/24) of the blastocysts had at least one chromosome with a deleted segment on the targeted DNA sequence (see an example in FIG. 8, Appendix to Nix et al., Proc. Natl. Acad. Sci. U.S.A. Nexus, 2023, 2(11), 1-14). In addition, 70% (17/24) of the blastocysts sequenced did not present a wild-type sequence in the targeted region (FIG. 2B, other two examples in FIG. 9, Appendix to Nix et al., Proc. Natl. Acad. Sci. U.S.A. Nexus, 2023, 2(11), 1-14. Applicant noted that 72 out of 89 blastocysts tested with Applicant's long-range oligos did not produce an amplicon, though the presence of DNA was confirmed by amplification of a non-targeted autosomal region in each sample. Therefore, Applicant can reason that these 72 blastocysts had unexpectedly larger DNA deletions (60, 61) that eliminated at least one oligonucleotide pairing site on all chromosomes. Under such reasoning, Applicant can estimate that 91% (81/89) of the blastocysts were edited with no wild-type sequence of the targeted DNA.

### Embryo Survival Following One or Two Cas9D10A Electroporation Sessions

Applicant tested if the electroporation of Cas9D10A with scramble gRNAs would impact development to the blastocyst stage. One electroporation session with scramble gRNAs produced similar results to controls (164-166 hpf—Cas9D10A and scramble gRNAs: 17.1% 3.1, controls: 25.3%±3.2; 188-190 hpf—Cas9D10A and scramble gRNAs: 31.5%±3.8, controls: 30.8%±3.4, P>0.05, Tables S1-S3, Appendix to Nix et al., Proc. Natl. Acad. Sci. U.S.A. Nexus, 2023, 2(11), 1-14). Two electroporation sessions with scramble gRNAs also produced similar results to controls (164-166 hpf—Cas9D10A and scramble gRNAs: 17.7% 2.6, controls: 25.3%±3.2; 188-190 hpf—Cas9D10A and scramble gRNAs: 28.2±3.1, controls: 30.8%±3.4, P>0.05, Tables S1-S3, Appendix to Nix et al., Proc. Natl. Acad. Sci. U.S.A. Nexus, 2023, 2(11), 1-14). Therefore, one or two electroporation sessions with Cas9D10A and scramble gRNAs did not reduce blastocyst yield and maintained survival like that seen in non-electroporated embryos.

TABLE S1
Blastocyst developmental rates for the experiments comparing one
versus two electroporation sessions with Cas9D10A and sgRNAs.

| | | single | | double | |
|---|---|---|---|---|---|
| | | electroporation | | electroporation | |
| | | N PZ | % ± SE | N PZ | % ± SE |
| 164- | Cas9D10A + targeting | 337 | 6.8 ± 1.4 | 655 | 3.2 ± 0.7 |
| 166 | sgRNAs | | | | |
| hpf | Cas9D10A + scramble | 146 | 17.1 ± 3.1 | 209 | 17.7 ± 2.6 |
| | sgRNAs | | | | |
| | Controls not | 182 | 25.3 ± 3.2 | 182 | 25.3 ± 3.2 |
| | electroporated * | | | | |
| 188- | Cas9D10A + targeting | 337 | 11.6 ± 1.7 | 655 | 7.9 ± 1.1 |
| 190 | sgRNAs | | | | |
| hpf | Cas9D10A + scramble | 146 | 31.5 ± 3.8 | 209 | 28.2 ± 3.1 |
| | sgRNAs | | | | |
| | Control not | 182 | 30.8 ± 3.4 | 182 | 30.8 ± 3.4 |
| | electroporated * | | | | |

For Table S1 * The drops used for control culture (not electroporated) were carried out in parallel with the experiments of two versus one electroporation and thus used on statistical tests for single electroporation and double electroporation. hpf: hours post fertilization; N PZ: number of putative zygotes; SE: standard error

### Table S2. Contrasts Between Experimental Groups for Blastocyst Yield at 164-166 Hpf.

TABLE S2
Contrasts between experimental groups for blastocyst yield at 164-166 hpf.

| contrast | odds ratio | SE | DF | null | t ratio | raw P | BH P |
|---|---|---|---|---|---|---|---|
| Cas9D10A 1×/Scramble 1× | 0.3545 | 0.1092 | 15 | 1 | −3.3657 | 0.0042 | 0.0073 |
| Cas9D10A 1×/Control | 0.2166 | 0.0596 | 16 | 1 | −5.5584 | <.0001 | <.0001 |
| Scramble 1×/Control | 0.6109 | 0.1699 | 10 | 1 | −1.7722 | 0.1068 | 0.0849 |
| Cas9D10A 1×/Cas9D10A 2× | 2.2114 | 0.6847 | 31 | 1 | 2.5633 | 0.0154 | 0.0218 |
| Cas9D10A 2×/Scramble 2× | 0.1540 | 0.0441 | 26 | 1 | −6.5322 | <.0001 | <.0001 |
| Cas9D10A 2×/Control | 0.0979 | 0.0274 | 26 | 1 | −8.3042 | <.0001 | <.0001 |
| Scramble 2×/Control | 1.5723 | 0.3913 | 11 | 1 | 1.8185 | 0.0963 | 0.0838 |

For Table S2—1×: one electroporation session; 2× two electroporation sessions; SE: standard error; DF: degrees of freedom; BH P: Bonferroni corrected P values.

TABLE S3
Contrasts between experimental groups for blastocyst yield at 188-190 hpf.

| contrast | odds ratio | SE | DF | null | t ratio | raw P | BH P |
|---|---|---|---|---|---|---|---|
| Cas9D10A 1×/Scramble 1× | 0.2845 | 0.0701 | 15 | 1 | −5.1005 | 0.0001 | 0.0002 |
| Cas9D10A 1×/Control | 0.2945 | 0.0689 | 16 | 1 | −5.2232 | 0.0001 | 0.0002 |
| Scramble 1×/Control | 1.0350 | 0.2483 | 10 | 1 | 0.1434 | 0.8888 | 0.8888 |
| Cas9D10A 1×/Cas9D10A 2× | 1.5176 | 0.3390 | 31 | 1 | 1.8676 | 0.0713 | 0.0998 |
| Cas9D10A 2×/Scramble 2× | 0.2192 | 0.0463 | 26 | 1 | −7.1936 | <.0001 | <.0001 |
| Cas9D10A 2×/Control | 0.1940 | 0.0419 | 26 | 1 | −7.5892 | <.0001 | <.0001 |
| Scramble 2×/Control | 1.1299 | 0.2512 | 11 | 1 | 0.5496 | 0.5936 | 0.6925 |

For Table S3—1×: one electroporation session; 2× two electroporation sessions; SE: standard error; DF: degrees of freedom; BH P: Bonferroni corrected P values.

One electroporation session with Cas9D10A and OCT4-targeting sgRNAs reduced the blastocyst yield relative to scramble or control groups (164-166 hpf—Cas9D10A and targeting sgRNAs: 6.8%±1.4, controls: 25.3% 3.2; 188-190 hpf—Cas9D10A and targeting sgRNAs: 11.6% 1.7, controls: 30.8% 3.4, P<0.001, Tables S1-3, Appendix of Nix et al., Proc. Natl. Acad. Sci. U.S.A. Nexus, 2023, 2(11), 1-14). Two electroporation sessions with Cas9D10A and OCT4-targeting sgRNAs also reduced blastocyst development (164-166 hpf—Cas9D10A and targeting sgRNAs: 3.2%±0.7, controls: 25.3%±3.2; 188-190 hpf—Cas9D10A and targeting sgRNAs: 7.9% 1.1, controls: 30.8% 3.4, P<0.001, Tables 51-3, Appendix to Nix et al., Proc. Natl. Acad. Sci. U.S.A. Nexus, 2023, 2(11), 1-14). Applicant also evaluated zygotes electroporated twice with Cas9D10A and OCT4-targeting sgRNAs (N=56), transferred into individual drops of media at the 8-cell stage and placed in a time-lapse incubator, along with controls that were not electroporated (N=28). A greater number of electroporated embryos arrested their development at the 8-cell (35.5% vs 17.8% controls, P=0.0013) and morula (51.8% vs 35.7% controls, P=0.0013) stages. Additionally, a lower proportion of the electroporated embryos developed to the blastocyst stage (12.5% vs 46.4% controls, P=1.06×10⁷, exact binomial test). Thus, targeting the gene OCT4 by two electroporation sessions of Cas9D10A and sgRNAs caused partial developmental arrest at the 8-cell and morula stages with a sharp decline in the development to the blastocyst stage but did not eliminate embryo survival.

mRNA Knockdown in Cattle Zygotes by Cas13a

To test whether Cas13a can target mRNAs in zygotes, first, Applicant electroporated PZ with exogenous mRNAs of fluorescent proteins (red (RFP) or green (GFP)). Fluorescence imaging of embryos ˜70 hpf showed successful introgression of exogenous mRNAs (GFP and RFP mRNAs) into PZ and expression of the corresponding proteins in cleavage embryos (FIG. 3). By contrast, Applicant quantified a significant reduction of fluorescence (1.37-fold for GFP, and 1.34-fold for RFP, P<0.001, FIG. 3) when Applicant electroporated PZ with the exogenous mRNA and Cas13a+targeting sgRNAs simultaneously. Since those PZ treated with Cas13a+targeting sgRNAs did not target an endogenous RNA, Applicant tested whether Cas13a RNPs would impact embryo development. There were no statistical differences in the development to blastocyst stage at 188-190 hpf (30.8%, 37.7%, 32.1%, 33.3% for Cas13a+GFP mRNA sgRNAs, Cas13a+RFP mRNA sgRNAs, Cas13a+scramble gRNAs, controls, respectively, P>0.8). Thus, Cas13a targets mRNAs efficiently in cattle zygotes with no alteration in their developmental potential.

### Ablation of OCT4 Function in Cattle Pre-Implantation Embryos by CRISPR-DART

Applicant used CRISPR-DART (FIG. 4) to target the promoter (based on orthology with the human genome) and exon 1 of OCT4. The induced deletions significantly reduced embryo survival (164-166 hpf—CRISPR-DART: 6.1%±0.8, controls: 23%±2.3; 188-190 hpf—CRISPR-DART: 13%±1.2, controls: 31.6%±2.5, P<0.001, Tables S4-S5, Appendix to Nix et al., Proc. Natl. Acad. Sci. U.S.A. Nexus, 2023, 2(11), 1-14). Using immunofluorescent staining, Applicant determined that the putatively edited blastocysts (Applicant estimated 91% editing success) did not produce OCT4 protein. Additionally, Applicant detected a decrease of NANOG in the edited blastocysts (FIG. 5A, see Supplemental Text, Appendix to Nix et al., Proc. Natl. Acad. Sci. U.S.A. Nexus, 2023, 2(11), 1-14). Thus, Applicant confirmed that the deletion of the promoter and exon 1 of OCT4 resulted in absence of OCT4 protein.

TABLE S4
Blastocyst developmental rates
for CRISPR-DART targeting OCT4.

| | | N PZ | % ± SE |
|---|---|---|---|
| 164- | DART + targeting sgRNAs | 830 | 6.1 ± 0.8 |
| 166 | DART + scramble gRNAs | 312 | 17.0 ± 2.1 |
| hpf | Controls not electroporated | 348 | 23.0 ± 2.3 |
| 188- | DART + targeting sgRNAs | 832 | 13.0 ± 1.2 |
| 190 | DART + scramble gRNAs | 312 | 30.1 ± 2.6 |
| hpf | Controls not electroporated | 348 | 31.6 ± 2.5 |

For Table S4—hpf: hours post fertilization; N PZ: number of putative zygotes; SE: standard error.

TABLE S5
Contrasts between experimental groups for blastocyst
yield in experiments with CRISPR-DART targeting OCT4

| | | odds | | | | | | |
|---|---|---|---|---|---|---|---|---|
| | contrast | ratio | SE | DF | null | t ratio | raw P | BH P |
| 164- | DART + targeting sgRNAs/ | 0.3199 | 0.0668 | 35 | 1 | −5.4566 | <.0001 | <.0001 |
| 166 | DART + scramble gRNAs | | | | | | | |
| hpf | DART + targeting sgRNAs/ | 0.2193 | 0.0423 | 37 | 1 | −7.8746 | <.0001 | <.0001 |
| | Control | | | | | | | |
| | DART + scramble gRNAs/ | 0.6855 | 0.1353 | 21 | 1 | −1.9129 | 0.0695 | 0.0834 |
| | Control | | | | | | | |
| 188- | DART + targeting sgRNAs/ | 0.3460 | 0.0556 | 35 | 1 | −6.6000 | <.0001 | <.0001 |
| 190 | DART + scramble gRNAs | | | | | | | |
| hpf | DART + targeting sgRNAs/ | 0.3228 | 0.0499 | 37 | 1 | −7.3100 | <.0001 | <.0001 |
| | Control | | | | | | | |
| | DART + scramble gRNAs/ | 0.9329 | 0.1575 | 21 | 1 | −0.4110 | 0.6852 | 0.6852 |
| | Control | | | | | | | |

For Table S5—SE: standard error; DF: degrees of freedom; BH P: Bonferroni corrected P values

Morphological examination showed an absence of a well-defined inner cell mass in blastocysts deemed OCT4^{-/-}, whereas a well-defined inner cell mass is clearly visible in the control embryos (FIG. 5B). Time-lapse image analysis of the development of putative OCT4^{-/-} embryos confirmed the formation of a blastocoel cavity and absence of a normal inner cell mass (Supplementary movies 1 and 2 of Nix et al., Proc. Natl. Acad. Sci. U.S.A. Nexus), 2023, 2(11), 1-14. Next, Applicant interrogated the transcriptome of OCT4−/− blastocysts. To confirm that the blastocysts collected were OCT4^{-/-}, Applicant obtained genomic DNA and total RNA from single embryos. Applicant used the genomic DNA to confirm the absence of the targeted DNA sequence (FIG. 5C) and evaluated the transcript abundance of 14156 protein-coding or long non-coding genes from five OCT4^{-/-} blastocysts. Comparative analyses revealed 125 genes with differential transcript abundance between OCT4^{-/-} blastocysts and controls (FIG. 5D, FDR<0.1). Eighty-three and 42 genes had lower and greater transcript abundance in OCT4^{-/-} blastocysts, respectively. Notably, 17 genes with differential transcript abundance were functionally related to the maintenance of pluripotency (see e.g., FIG. 5E for examples). These results indicate that a blastocoel cavity may form in the absence of OCT4, but the formation of the inner cell mass and normal gene expression is severely impaired in cattle OCT4^{-/-} embryos.

### Discussion

Applicant developed an approach using Cas9D10A to delete targeted regions of the DNA and Cas13a to cleave targeted RNA for complete disruption of gene function in cattle zygotes at high efficiency. Applicant used CRISPR-DART to target OCT4 mRNAs and exon 1, including the promoter region. Applicant's data provide several insights into the function of OCT4 in cattle pre-implantation development. First, most OCT4^{-/-} embryos arrest development before the blastocyst stage, but a minor proportion of edited zygotes do still survive. Second, OCT4^{-/-} embryos that progress their development are able to form a blastocoel cavity with an outer layer of cells resembling trophectoderm but do not form an inner cell mass with similar morphology observed in control embryos. Finally, the ablation of OCT4 significantly alters the transcript abundance of genes involved in pluripotency. Applicant's results show that OCT4 is necessary for the development of a cattle blastocyst with a morphologically normal inner cell mass.

### Simultaneous Deletion of DNA Segments and Cleavage of RNA in Zygotes

Previous research has reported the use of CRISPR-Cas9 to delete DNA segments in cattle zygotes (32, 35-37, 40). To build on this, Applicant tested the efficacy of Cas9 and Cas9D10A with two sgRNAs targeting the exon 1 of OCT4. Although Applicant did not test for off-targets, Cas9D10A produces single-strand DNA breaks and requires two sgRNAs targeting opposite strands to nick the DNA and induce faulty DNA repair (62). This combination of factors significantly reduces mutation elsewhere in the genome. Applicant's results confirmed that Cas9D10A RNPs produce large deletions beyond the region flanked by the sgRNAs (63). However, Applicant only detected deletions larger than 500 nucleotides when Applicant electroporated the zygotes twice in an interval of six-hours between sessions. Two electroporation sessions allow for the introduction of greater quantities of RNPs in the zygote without causing toxicity (64). The combination of Cas9D10A targeting two sequences in the genome, and likely a higher quantity of RNPs entering the cell in two sessions of electroporation, increased the efficiency in producing full edits from 25.9 to 91%, which is higher than previous reports in cattle zygotes (32, 35-37, 40).

The RNPs produced by the combination of CRISPR-Cas13a and an sgRNA can target and cleave single stranded RNAs (65, 66). These RNPs have been used in animal embryos including in mouse (67) and pig (67) to target specific mRNAs. Here, Applicant tested the efficacy of Cas13a in cattle zygotes by introducing and targeting mRNAs for either GFP or RFP. Applicant's experiments showed that Cas13a could efficiently prevent protein synthesis from targeted mRNAs in cattle zygotes. One concern related to Cas13a is that it may cleave unintended mRNAs in the vicinities of targeted RNAs in a cell-dependent manner (68). The introduction of Cas13a+sgRNAs targeting exogenous mRNAs (GFP or RFP) and the corresponding mRNAs into cattle zygotes did not reduce embryo survival, thus, if there are off-target effects, they are negligible in cattle pre-implantation embryos. Cas13a can knockdown specific mRNAs in zygotes in conjunction with Cas9D10A to target genomic DNA.

### Effects of Ablation of OCT4 in Cattle Embryos

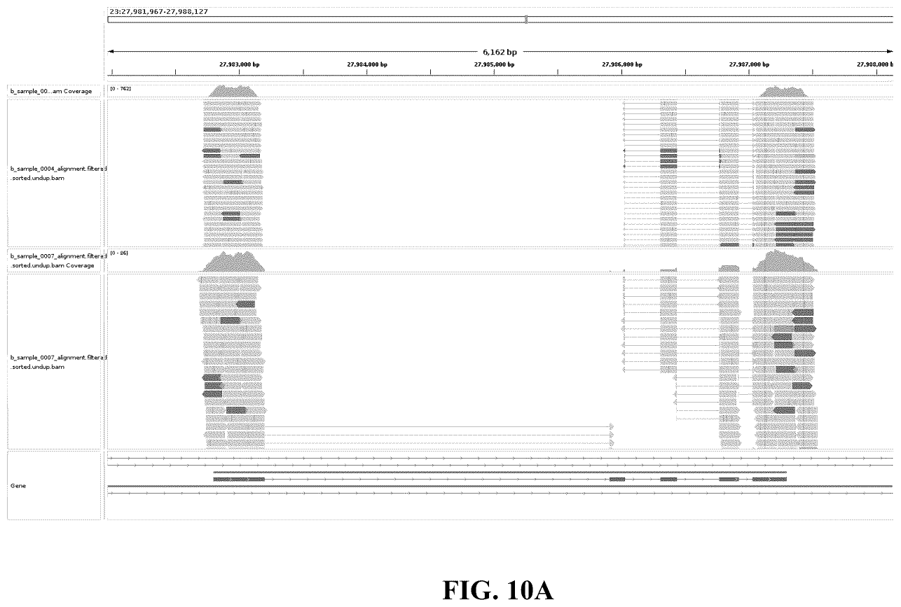

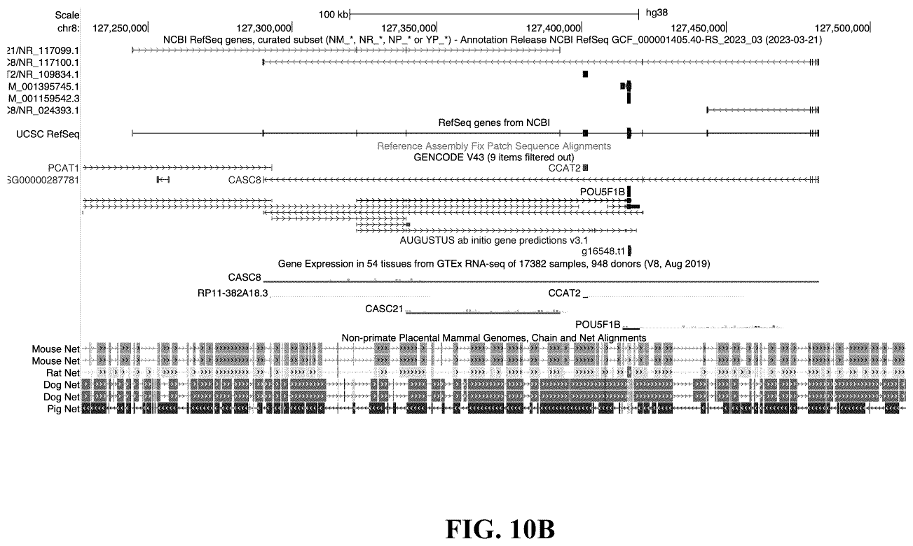

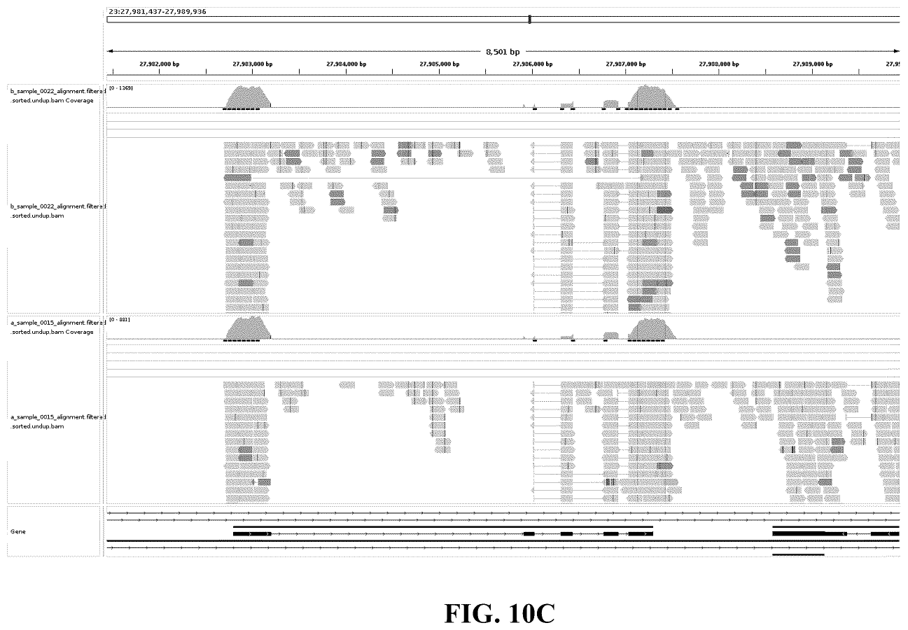

Applicant's CRISPR-DART approach efficiently deleted exon 1 and the promoter from the OCT4 in most embryos. Applicant hypothesized that removing the promoter and transcript starting site would impair the production of OCT4 mRNAs and proteins. Applicant confirmed that most of the embryos tested by immunofluorescence assays did not have detectable OCT4. However, Applicant's RNA-sequencing data, produced from confirmed OCT4^{-/-} embryos, showed sequences aligning with all five exons of OCT4 (FIG. 10A, Appendix of Nix et al., Proc. Natl. Acad. Sci. U.S.A. Nexus), 2023, 2(11), 1-14). Both data are conflicting, but Applicant reasons that a pseudogene of OCT4 produced RNAs that were sequenced and mapped to the OCT4 gene. Few lines of evidence support Applicant's rationale. Applicant only selected sequences that mapped to the cattle reference genome once to quantify transcript abundance. Messenger RNAs produced by this pseudogene would have mapped to both genomic regions, but this region is not present in the current cattle reference genome, as indicated by the comparative mapping in the UCSC genome browser (FIG. 10B, Appendix of Nix et al., Proc. Natl. Acad. Sci. U.S.A. Nexus), 2023, 2(11), 1-14). Annotated OCT4 pseudogenes are long non-coding RNAs (69-74) and do not contain introns (FIG. 10A, Appendix of Nix et al., Proc. Natl. Acad. Sci. U.S.A. Nexus), 2023, 2(11), 1-14). Lastly, one hallmark observation in Applicant's data is that no sequence mapped to OCT4 intronic regions in edited embryos (FIG. 10B, Appendix of Nix et al., Proc. Natl. Acad. Sci. U.S.A. Nexus), 2023, 2(11), 1-14), whereas several intronic sequences from all introns were evident in the control embryos (FIG. 10C, Appendix of Nix et al., Proc. Natl. Acad. Sci. U.S.A. Nexus), 2023, 2(11), 1-14)). Thus, Applicant concluded that the RNA sequences mapping to the annotated OCT4 cattle gene for OCT4^{-/-} embryos were transcribed from a pseudogene with no intron. This confounding factor between functional OCT4 and pseudogenes has been long observed in stem cell research (71).

The ablation of OCT4 function in cattle pre-implantation embryos severely reduced blastocyst development, but a majority of the blastocysts were confirmed to be fully edited. This finding aligns with reports that produced embryos from OCT4^{-/-} somatic cells (56, 57) or produced putative OCT4^{-/-} embryos by introducing RNPs into zygotes (35, 36). The major blastocyst phenotype Applicant observed was the absence of an inner cell mass, a phenotype previously reported in knockout mice (Pou5f1^{tm1Cgre}/Pou5f1^{tm1Cgre}, genotype id MGI:3040797 (49)). By comparison, Simmet and colleagues showed that OCT4 is necessary only for the second lineage differentiation (57), and it is possible that dysregulated genomic reprogramming due to somatic cell cloning could be the cause of the minor discrepancy in the phenotypes. Applicant's results show that OCT4 is required for the differentiation of inner cell mass in cattle embryos.

Applicant also evaluated the transcriptomic profile of OCT4^{-/-} blastocysts. Applicant's results, contrasting the transcriptome of OCT4^{-/-} in vitro produced blastocysts and controls, showed 125 genes with significant alteration in transcript abundance. Only seven genes overlapped with the dataset reported elsewhere (ARF5, MT1E, NUPR1, PLA2G15, RRM2, STAT3, and SWAP70 (57)), but all seven genes had the same direction of altered transcript abundance. Among the genes up-regulated in OCT4^{-/-} blastocysts, MT1E potentiates cell differentiation (75, 76), and NUPR1 is activated when the cells are under oxidative stress (77). Conversely, among the genes with lower transcript abundance in OCT4′ blastocysts, RRM2 is a known marker of pluripotent stem cells (78), STAT3 is required for embryonic stem cell pluripotency (79), and the absence of SWAP70 impairs the self-renewal of mouse hematopoietic stem cells (80).

Applicant's results also highlighted a dysregulation in other genes with roles in regulating stem cell function. For example, DPPA4 and CADHD1 are pluripotent stem cell markers (78) and have a lower abundance of transcripts in OCT4^{-/-} blastocysts. Also, with a lower abundance of transcripts in edited embryos, ZIC2 (81, 82), KLFI7 (83, 84), FOXD3 (85), HDAC8 (85), and GNL3 (86) are directly involved in the regulation of pluripotency. By contrast, ADAM9 (87), ANXA8L1 (87), CDKN1A (88), DDAH2 (89), ELF3 (89), HOXC10 (90) and LGI1 (90) are all up-regulated in OCT4′ blastocysts and promote cellular differentiation. Collectively, these results show a severe imbalance in the regulation of genes associated with stemness or cell differentiation, which is coherent with the absence of an inner cell mass in OCT4^{-/-} blastocysts.

### Conclusion

The production of knockouts is essential for mechanistic studies of gene function in pre-implantation embryos. Applicant showed that Cas9D10A is more efficient than Cas9 at producing biallelic deletions in zygotes. Two sessions of electroporation introduce greater quantities of Cas9D10A RNPs and increase the frequency of large biallelic deletions. The sequential introduction of RNPs does not impair embryo development as long sgRNAs targeting proximal sequences in the genome are not used. RNPs consisting of Cas13a prevent protein production from targeted mRNAs in cattle zygotes. Applicant's CRISPR-DART approach increased the efficiency of producing knockout zygotes. Lastly, Applicant showed that OCT4 is required for the regulation of several genes that control pluripotency and the formation of an inner cell mass in cattle blastocysts.

### Materials and Methods

### In Vitro Production of Embryos

Unless otherwise specified, all reagents were purchased from Sigma-Aldrich.

All procedures and culture media composition for in vitro production of embryos are described in detail elsewhere (94, 95). Briefly, Applicant obtained cattle ovaries from an abattoir (Brown Packing, Gaffney, SC) and washed them with anti-biotic anti-mycotic (Antibiotic-Antimycotic 100×, Thermofisher Scientific, Waltham, MA) and 0.9% saline solution. For the collection of cumulus-oocyte-complexes (COCs), Applicant aspirated ovarian follicles 3-8 mm in diameter using an 18 g needle (Single-Use Needles BD Medical, VWR, Philadelphia, PA) connected to a regulated vacuum system and collection bottle containing oocyte collection medium (OCM, BoviPlus Oocyte Collection Medium, Minitube, Verona, WI) supplemented with gentamicin (50 μg/μl) and heparin (2 U/ml). Applicant washed COCs twice in OCM, followed by three washes in oocyte maturation medium (OMM). Then Applicant selected COCs with homogeneous, non-granular oocyte cytoplasm and three or more compact layers of cumulus for in vitro maturation. COCs were placed in groups of 10 in 50 μl of OMM covered by light mineral oil. In vitro maturation plates were incubated for 22-24 hours at 38.5° C. and 5% CO₂ humidified atmosphere. Following the incubation, Applicant washed the mature COCs in synthetic oviductal fluid medium (SOF) containing N-2-hydroxyethylpiperazine-N′-2-ethanesulfonic acid (HEPES-TL, Thermofisher Scientific, Waltham, MA) and SOF for fertilization (SOF-FERT) before transferring into a final fertilization plate (100 COCs/ml). Applicant thawed frozen semen straws and processed sperm prior to transfer into fertilization plates at a concentration of 1,000,000 spermatozoa/ml. COCs and spermatozoa were co-incubated for 12-13 hours under the same conditions described for in vitro maturation.

Applicant removed putative zygotes (PZ) from fertilization medium at approximately 14 hours post fertilization (hpf) and denuded the cumulus cells by vortexing in 1% hyaluronidase for 5 minutes. Next, Applicant moved PZ through three washes of SOF-HEPES and SOF culture medium (SOF-BE1). The PZs used for control groups were placed in their final culture dish immediately after the washes. Alternatively, the PZs used for electroporation were placed in temporary culture dishes containing 50 μl SOF-BE1 covered with light mineral oil. After electroporation, Applicant washed the PZs in SOF-BE1 before placing them in culture. PZs were cultured in groups of 25-30 in 50 μl SOF-BE1 covered by light mineral oil, incubated at 38.5° C. with 5% CO₂, 5% O₂ in a humidified Eve Benchtop Incubator (WTA, College Station, TX).

For time-lapse image analysis, Applicant cultured 8-cell embryos individually in 15 μl SOF-BE1 covered by light mineral oil, incubated at 38.5° C. with 5% CO₂ and 5% O₂ in a MIRI Time-Lapse Incubator (Esco Medical, Egaa, DK).

### Guide RNA Design

Applicant designed sgRNAs to target the genomic DNA of the transcriptional start site and exon 1 of OCT4 using the CRISPOR webservice (96). Applicant designed the sgRNAs for Cas13a using New York Genome's cas13 design tool (97, 98) to target the 4th exon of the OCT4 mRNA. As an independent layer of in silico validation, Applicant aligned all sgRNAs targeting the OCT4 gene or transcript to the bovine genome with the BLAT software in the UCSC Genome Browser (99). Additionally, Cas13a sgRNAs were designed to target CleanCap EGFP and mCherry mRNAs (5moU, TriLink Biotechnologies, San Diego, CA). The targeting sgRNAs used in this study were OCT4 sgRNA1: CTTCGCCTTCTCGCCCCCGCCGG (SEQ ID NO: 20), OCT4 sgRNA2: TGTCCCGCCATGGGGAAGGAAGG (SEQ ID NO: 21), OCT4 mRNA sgRNA: ATGCTCTCCAGGTTGCCTCT (SEQ ID NO: 22), mCherry mRNA sgRNA: TCCTCGAAGTTCATCACCCG (SEQ ID NO: 23), EGFP mRNA sgRNA: CATGATATAGACGTTGTGG (SEQ ID NO: 24). Applicant purchased all sgRNAs as a single RNA molecule comprised of both crRNA and tracrRNA sequences (Integrated DNA Technologies (IDT), Research Triangle Park, NC). Applicant also purchased a scramble gRNA (Alt-R® CRISPR-Cas9 Negative Control crRNA #1) and tracrRNA (Alt-R® CRISPR-Cas9 tracrRNA) from IDT and combined them following the manufacturer's instructions.

### Preparation of Ribonucleoprotein and Procedures for Electroporation

Applicant mixed Cas9 and sgRNAs for the formation of ribonucleoproteins in Opti-MEM™ Reduced Serum Medium (Thermofisher Scientific, Waltham, MA), and maintained the solution at room temperature for at least 30 minutes prior to electroporation. The specific concentrations and enzymes are detailed below.

As detailed above for control cultures, Applicant removed the cumulus cells from the PZ and placed them in holding SOF-BE1 at 38.5° C., 5% CO₂, and 5% O₂. Applicant removed PZs in groups of 30-40 from a holding culture and briefly washed them in OptiMEM (previously equilibrated in the incubator at 38.5° C. and 5% CO₂). Next, Applicant mixed 3 μl of the solution containing ribonucleoproteins with 3 μl of OptiMEM containing PZs. Applicant carried out the electroporation using a BTX oocyte petri dish with platinum electrodes (Harvard Apparatus, VWR, Philadelphia, PA). Applicant transferred the final 6 μl to the electroporation chamber. Impedance was checked and, if necessary, adjusted to measure between 0.19 and 0.20 by the addition of OptiMEM or removal of the electroporation solution. The electroporation parameters were as follows: six poring pulses of 15 volts, with 10% decay, for two milliseconds with a 50-millisecond interval, immediately followed by five transfer pulses of 3 volts, 40% decay, for 50 milliseconds with a 50-millisecond interval, alternating the polarity. Following the electroporation, Applicant washed the PZ with OptiMEM and SOF-BE1.

### Cleavage Assay of the Targeted DNA

Applicant carried out a cleavage assay to assess the formation and cleavage of DNA by ribonucleoproteins (100). Applicant amplified a segment of genomic cattle DNA to be targeted by the sgRNAs by assaying a PCR using the following oligonucleotides (forward: GGCAAGGAACTTGATGCACG (SEQ ID NO: 25) and reverse: TGGCCAACCCACTGTTTGAT (SEQ ID NO: 26)). The PCR reaction mix consisted of 0.2 IU/μl Phusion Hot Start II DNA Polymerase (Thermofisher Scientific, Waltham, MA), 1× Phusion HF Buffer, 200 μM dNTPs (Promega, Madison, WI), and forward and reverse oligonucleotides (IDT, Coralville, Iowa) at 0.10 μM each, in a final volume of 20 μl in 0.2 ml clear PCR tubes. The cycling conditions for this reaction were: 98° C. for 1 minute, followed by 40 cycles of 98° C. for 15 seconds, 55° C. for 45 seconds, and 72° C. for 1 minute, followed by a final extension of 4 minutes at 72° C.

Applicant incubated Cas9 (1 μM, Integrated DNA Technologies, Research Triangle Park, NC) with either sgRNA1 or sgRNA2 300 nM in OptiMEM for 30 minutes at room temperature to form the RNPs. Next, Applicant incubated RNPs with DNA fragments containing the targeted sequence (1:10 (v:v) Cas9+sgRNA, 3 nM DNA, 1×NEB buffer 3.1) at 37° C. for 3 hours. Fragments were assessed by electrophoresis on a 1.5% Agarose I™ gel followed by staining with Diamond™ Nucleic Acid Dye and imaging.

### Evaluation of Electroporation Efficiency

Applicant evaluated the electroporation efficiency with RNPs formed by Cas9-RFP (Alt-R™ S.p.Cas9-RFP V3, Integrated DNA Technologies, Research Triangle Park, NC) at 800 ng/μl and scramble gRNAs at 800 ng/μl. After washing the PZs in OptiMEM, Applicant imaged them using a fluorescent microscope (details below).

### Assessment of Sequence Deletions by Cas9 or Cas9D10A

To test the pattern of deletions with either a double-cutting enzyme or a nickase, Applicant carried out a single electroporation at approximately 15 hpf with ribonucleoproteins formed by either Cas9 or Cas9D10A (IDT) at 800 ng/μl and sgRNAs at 800 ng/μl each. After washing the PZ in SOF-BE1, Applicant placed them in culture as described for control PZs.

Assessment of mRNA Cleavage by Cas13a

Applicant carried out a single electroporation of PZs with one of the following solutions: a) mRNA of either mCherry or GFP at 400 ng/μl; or b) mRNA of either mCherry or GFP at 400 ng/μl and ribonucleoprotein formed by Cas13a (GenScript, Piscataway, NJ) at 400 ng/μl and the corresponding targeting sgRNA at 400 ng/μl. After washing the PZ in OptiMEM, Applicant imaged them using a fluorescent microscope (details below) in SOF-HEPES.

### CRISPR-DART

For CRISPR-DART, Applicant carried out the first electroporation at approximately 14 hpf with 3 μl of RNPs formed by Cas9D10A at 600 ng/μl and sgRNAs at 800 ng/μl each mixed with 3 μl of OptiMEM. The PZs were maintained in SOF-BE1 media in the incubator. Then, Applicant electroporated them again at approximately 20 hpf with two solutions of RNP complexes prepared separately. One solution contained Cas9D10A at 600 ng/μl and each sgRNA at 800 ng/μl and the other contained Cas13a at 1600 ng/μl and sgRNA at 800 ng/μl. At the time of electroporation, Applicant mixed 1.5 μl of each RNP with 3 μl of OptiMEM containing the PZ. After washing the PZ in SOF-BE1, Applicant placed them in culture as described for control PZs.

### Targeted DNA Sequencing

All embryos collected for DNA sequencing were washed in PBS 0.1% BSA fraction V, followed by removal of the zona pellucida by exposure to EmbryoMax® Acidic Tyrode's Solution and gentle pipetting. Once the zona pellucida was removed, Applicant washed the embryos in PBS 0.1% BSA fraction V twice and collected them individually in microtubes in approximately 1 μl PBS 0.1% BSA fraction V. Applicant exposed the nucleic acids of each embryo with 5 μl of QuickExtract™ DNA Extraction Solution (Lucigen, VWR, Philadelphia, PA), and incubated at 65° C. for 15 minutes followed by 2 minutes at 98° C.

### High Throughput Short Reads

Applicant assayed a PCR using oligonucleotides flanking the targeted deletion site with coupled sequencing adapters on their 5′ end (forward: acactctttccctacacgacgctcttccgatctAGAGGTGTTGAGCAGTCTCTAGG (SEQ ID NO: 27), reverse: gtgactggagttcagacgtgtgctcttccgatctGTAGGCCATCCCTCCACAC (SEQ ID NO: 28); lower case letters indicate adapter, uppercase letters indicate targeted sequence). The PCR reaction consisted of 0.2 IU/μl Phusion Hot Start II DNA Polymerase (Thermofisher Scientific, Waltham, MA), 1× Phusion HF Buffer, 200 μM dNTPs (Promega, Madison, WI), and oligonucleotides (IDT) at 0.1 μM each, in a final volume of 20 μl. Reactions were carried out in 0.2 ml clear PCR tubes (VWR, Philadelphia, PA), and the cycling conditions were: 98° C. for 1 minute, followed by 40 cycles of 98° C. for 15 seconds, 61° C. for 30 seconds, and 72° C. for 40 seconds, proceeding a final extension of 4 minutes at 72° C. Applicant confirmed the amplification using 2% Agarose I™ (VWR, Philadelphia, PA) and gel electrophoresis, followed by DNA staining with Diamond™ Nucleic Acid Dye (Promega, Madison, WI).

Next, Applicant completed the library preparation with a second PCR using oligonucleotides obtained from xGen™ UDI Primers Plate 1, 8 nt (IDT). The reaction mixture consisted of 0.3 IU/μl Phusion Hot Start II DNA Polymerase (Thermofisher Scientific, Waltham, MA), 1× Phusion HF Buffer, 200 μM dNTPs (Promega, Madison, WI), and 3 μM Illumina adaptors, in a final volume of 25 μl. The reaction was assayed according to the following conditions: 98° C. for 30 seconds, followed by 15 cycles of 98° C. for 10 seconds, 60° C. for 30 seconds, and 72° C. for 30 seconds, proceeding a final extension of 5 minutes at 72° C. Applicant pooled the amplicons and size-selected the targeted fragments using a 2% Invitrogen™ UltraPure™ Low Melting Point Agarose gel (Fisher Scientific, Waltham, MA) followed by a purification using the Zymoclean Gel DNA Recovery Kit (Zymo Research, Irvine, CA). The libraries were sequenced at the Vanderbilt Technologies for Advanced Genomics using a NovaSeq 6000 System (Illumina, Inc, San Diego, CA) to produce pair-end reads 150 nucleotides long.

Applicant processed the fastq files using an in-house bioinformatic pipeline similar to one published elsewhere (101). Applicant only proceeded with reads #2 because it spanned Applicant's targeted DNA region. First, Applicant used trimmomatic v.0.39 (102) to remove the sequencing adapters and filtered reads to retain those with a minimum length of 100 nucleotides and a minimum average quality score of 35. Then Applicant used clumpify.sh from BBTools (https://sourceforge.net/projects/bbmap/) to remove duplicates. Lastly, Applicant converted the file format from fastq to fasta using seqtk (103).

### High Throughput Ion Reads

Applicant produced amplicons by assaying a PCR using the following oligonucleotides (forward: GGCAAGGAACTTGATGCACG (SEQ ID NO: 25) and reverse: TGGCCAACCCACTGTTTGAT (SEQ ID NO: 26)). The PCR reaction mix consisted of 0.2 IU/μl Phusion Hot Start II DNA Polymerase (Thermofisher Scientific, Waltham, MA), 1× Phusion HF Buffer, 200 μM dNTPs (Promega, Madison, WI), and forward and reverse oligonucleotides (IDT, Coralville, Iowa) at 0.10 μM each, in a final volume of 20 μl in 0.2 ml clear PCR tubes. The cycling conditions for this reaction were: 98° C. for 1 minute, followed by 40 cycles of 98° C. for 15 seconds, 55° C. for 45 seconds, and 72° C. for 1 minute, followed by a final extension of 4 minutes at 72° C. Applicant confirmed the amplification by assaying 5 μl of each amplicon by electrophoresis on a 1.5% Agarose I™ gel before staining with Diamond™ Nucleic Acid Dye and imaging. When the amplification produced an amplicon, Applicant used the remaining PCR products to prepare sequencing libraries with the Native Barcoding Kit 24 V14 (Oxford Nanopore Technologies, Lexington, MA) following the manufacturer's instructions. Applicant sequenced the libraries on a MinION Mk1C (Oxford Nanopore Technologies, Lexington, MA).

Applicant processed the fast5 files with Guppy (v 6.4.2) (104) using the configuration file dna_r10.4.1_e8.2_260bps_sup.cfg for super high accuracy base calling. Next, Applicant used porechop (https://github.com/rrwick/Porechop) to remove adapters and used Fitlong (https://github.com/rrwick/Filtlong) to remove sequences smaller than 500 nucleotides long and with a quality of less than 90%. Applicant aligned the remaining sequences to the cattle reference genome (ARS-UCD1.2) using minimap2 (v 2.24) (105), allowing for spliced alignment (parameters: -ax map-ont --splice -c --cs=long --secondary=no --sam-hit-only -Y --splice-flank=no -G2k). Finally, Applicant used samtools (106) to remove alignments with less than 500 nucleotides mapped to the genome and supplementary alignments.

### Sanger Sequencing

Applicant produced PCR amplicons using the procedures described for “High throughput long reads. When the amplification produced an amplicon, Applicant treated the remaining PCR products with 3 μl ExoSAP-IT™ Express PCR Product Cleanup Reagent (Thermofisher Scientific, Waltham, MA) and incubated at 37° C. for 15 minutes followed by 80° C. for 15 minutes. The sequencing assay was carried out by the Genomics Sequencing Center at Virginia Tech using the same forward oligonucleotide used for the initial PCR.

### DNA and RNA Sequencing of Single Embryos

Applicant collected embryos for DNA and RNA-sequencing on stage codes six or seven (107). Applicant washed in PBS 0.1% BSA fraction V, followed by removal of the zona pellucida by exposure to EmbryoMax® Acidic Tyrode's Solution and gentle pipetting. Once the zona pellucida was removed, Applicant washed the embryos in PBS 0.1% BSA fraction V twice and collected them individually in microtubes in approximately 1 μl PBS 0.1% BSA fraction V. Applicant placed the tubes on dry ice and stored the samples at −80° C.

Applicant lysed the embryos by adding 10 μl of lysis solution, consisting of: 8.3 μl Luna Cell Ready Lysis Buffer 2× (New England Biolabs, Ipswich, MA), 0.66 μl Luna Cell Ready RNA Protection Reagent 25× (New England Biolabs, Ipswich, MA), 0.66 μl Luna Cell Ready Protease 25×, (New England Biolabs, Ipswich, MA), and 0.33 μl RNasin® Plus Ribonuclease Inhibitor (40 U/μl) (Promega, Madison, WI). Applicant incubated the solution on ice for 10 minutes, mixed by pipetting, then split the solution into two tubes. One tube, dedicated to DNA sequencing was further incubated at 37° C. for 15 minutes, followed by the addition of one μl of Luna Cell Ready Stop Solution 10× (New England Biolabs, Ipswich, MA). Applicant extracted DNA (108) by adding a solution of Sodium Acetate (5M) for a final concentration of 2.5 M, followed by the addition of 150 μl of Ethanol 100%. Applicant stored the solution −20° C. for over 15 hours and precipitated the DNA by centrifugation at 15,000×g for 20 minutes at 4° C. Applicant washed the pellet twice with 150 μl Ethanol 70% and eluted it with nuclease-free water. The DNA was used as template for amplification of the targeted region using the oligonucleotides and procedures for “High throughput long reads”. Preparation of libraries, sequencing and processing of sequences were carried out as described above.

Applicant extracted total RNA and from each 1%2 blastocyst using TRIzol reagent with Phasemaker Tubes for enhanced RNA purity and yield (109-112). RNA was stored in 70% ethanol at −80° C. (109) until library preparation. Applicant assessed RNA integrity of samples not used for sequencing with a 2100 Bioanalyzer Instrument (Agilent, Santa Clara, CA) and RNA 6000 Pico Kit (Agilent, Santa Clara, CA). These tests require the total volume of extracted RNA, therefore, only test samples were assessed to ensure the quality and rigor of Applicant's procedures. Applicant amplified cDNA using a modified mcSCRB-seq protocol and produced libraries using the Illumina DNA Prep kit (Illumina, Inc, San Diego, CA) (109, 113). The libraries were sequenced at the Vanderbilt Technologies for Advanced Genomics using a NovaSeq 6000 System (Illumina, Inc, San Diego, CA) to produce approximately 30 million pair-end reads 150 nucleotides long per sample.

Applicant aligned the RNA-sequencing data to the cattle reference genome (114) (ARS-UCD1.2/bosTau9) obtained from the Ensembl database (115, 116) using HISAT2 (v2.2.1) (117), followed by filtering with samtools (v1.17) (106, 118) to remove alignment less than 100 nucleotides long and with more than 5% mismatch nucleotides, plus removal of duplicates with biobambam2 (v2.0.95) (119). Next, Applicant counted the sequences matching the reference annotation (Bos_taurus.ARS-UCD1.2.105.gtf) using featureCounts (v2.0.1) (120).

### Statistical Analyses

The analytical procedures used to analyze differences in embryo development and differential transcript abundance, including the supplementary tables and pertinent graphs are available at: https://biase-lab.github.io/crispr_dart/

### Assessment of Differences in Embryo Development

Applicant recorded the number of embryos that developed to the blastocyst stage and the number of putative zygotes with arrested development prior to blastocyst formation at 164-166 hpf and 188-190 hpf for each culture drop. Culture drop was considered biological replicate. Applicant analyzed count data (success of blastocyst development or developmental arrest) using a general linear model with a binomial family, which results in logistic regression analysis, using the “glm” function from the R package “stats”. Applicant used the number of blastocysts and the number of putative zygotes that failed to develop into blastocysts as the dependent variable, and the group (control, scramble, or Cas treated) was a fixed effect. The Wald statistical test was conducted with the function “Anova” from the R package “car” (121). Finally, Applicant carried out a pairwise comparison using the odds ratio and two-proportion z-test employing the “emmeans” function of the R package “emmeans”. The null hypothesis assumed that the odds ratio of the proportion (p) of two groups was not different from 1 (H₀: p₁/p₂=1). Applicant adjusted the nominal P value for multiple hypothesis testing with the Bonferroni approach and inferred significance when adjusted P value<0.05.

Applicant analyzed data obtained from single embryo culture, with each embryo as a biological replicate, using the exact binomial test in R with the function “binom.test” (122). Significance was inferred if the P value<0.05.

### Assessment of Differences in Fluorescence

First, Applicant calculated corrected total cell fluorescence (CTCF) using the standard formula: Integrated Density−(Area of selected cell×Mean fluorescence of background readings). Applicant obtained the measurements necessary for the formula using the NIS-elements Imaging Software (v.5.02). Next, Applicant fitted a linear model using the “lm” function of the R package “stats” where Log 2(CTCF) was the dependent variable. Replicate and group (fluorescence protein mRNA or fluorescence protein mRNA+Cas13a and targeting sgRNA) were included as fixed effects. Applicant assessed the significance of the variables using the “Anova” function of the R package “car”. Next, Applicant tested the pair-wise significance of the two groups by a t-score test employing the “emmeans” function of the R package “emmeans”. The null hypothesis assumed that the difference between two averages (x̅) was not different from zero (H₀: x̅₁−x̅₂=0), and significance was inferred at alpha=0.05.

### Differential Transcript Abundance

In R software (123, 124), Applicant created one matrix with the read counts for all samples and retained genes classified as protein-coding and long non-coding DNA for downstream analysis. Applicant calculated counts per million (CPM) using the function ‘cpm’ from the R package ‘edgeR’ (125) and retained genes if CPM>1 in 4 or more samples. Applicant also calculated transcript per million as described elsewhere (126, 127) used for plotting the data. Applicant estimated differential transcript abundance between edited and control blastocysts employing the quasi-likelihood negative binomial generalized log-linear model from the R package “edgeR” (125) and the Wald test from the R package “DESeq2” (128). Applicant inferred statistically significant differences when False Discovery Rate (129) was less than 0.1 in both tests.

### Data Availability

The transcriptome data produced in this research is publicly available in the Gene Expression Omnibus repository under the accession GSE236474.

### Supplemental Text

### Immunofluorescence of OCT4 and NANOG

Applicant thinned each blastocyst's zona pellucida by brief immersion in EmbryoMax® Acidic Tyrode's Solution. Embryos were then washed and fixed in 4% paraformaldehyde solution for 15 minutes at room temperature. Next, Applicant washed the embryos before transfer into permeabilization solution (0.25% Triton X-100) for 30 minutes, then blocking solution (10% horse serum, Thermofisher Scientific, Waltham, PA), for 1 hour at room temperature. Applicant carried out concurrent immunofluorescence assay for OCT4 and NANOG proteins by incubation of embryos and antibodies conjugated to fluorescence dyes (mouse OCT4-alexafluor594 monoclonal antibodies (sc-5279) (130, 131) and mouse NANOG-alexafluor488 monoclonal antibodies (sc-374001) (132), Santa Cruz Biotechnology, Dallas, TX) at 4° C. for 24 hours and room temperature for 1 hour, respectively. Following washes, Applicant placed the embryos in DAPI solution for 5 minutes. Embryos were individually placed into 5 μl droplets of phosphate buffered solution (Thermofisher Scientific, Waltham, PA) submerged in mineral oil on a chambered coverglass (Thermofisher Scientific, Waltham, PA) for imaging.

### Fluorescence Imaging

Applicant evaluated fluorescence in Applicant's PZs or blastocysts using a Nikon Ti Eclipse fluorescence microscope (Nikon) coupled to X-Cite 120 epifluorescence illumination system and a DS-L3 digital camera using the cubes for DAPI (ex: 340-380 nm, em: 435-485 nm), alexafluor488 (ex: 465-495 nm, em: 515-555 nm) or alexafluor594 (ex: 532-587 nm, em: 608-683 nm). The microscope was controlled by NIS-elements Imaging Software (v.5.02).

### Mapping and Graphical Visualization of DNA Sequences Relative to the Bovine Genome

For visualization and graphical representation of the edited sequences, Applicant mapped resulting sequences in the fasta format to the cattle reference genome (ARS-UCD1.2) using the blat program (133) embedded in the UCSC genome browser (134, 135).

### PCR of a Non-Targeted Region

For all samples failing to amplify with primers used targeted sequencing, Applicant performed a PCR reaction targeting a segment of gene CDK1 using the following oligonucleotides: GCCCAGACCCAGCATCATT (SEQ ID NO: 29), GGGAGTGCCCAAAGCTCTAAA (SEQ ID NO: 30) (IDT). The reaction mix consisted of 0.2 IU/1 Phusion Hot Start II DNA Polymerase (Thermofisher Scientific, Waltham, MA), 1× Phusion HF Buffer, 200 μM dNTPs (Promega, Madison, WI), and forward and reverse oligonucleotides at 0.10 μM each, in a final volume of 20 μl in 0.2 ml clear PCR tubes. The cycling conditions for this reaction were: 98° C. for 1 minute, followed by 40 cycles of 98° C. for 15 seconds, 60° C. for 30 seconds, and 72° C. for 45 seconds, before a final extension of 4 minutes at 72° C. To check for the presence of DNA, 5 μl of each PCR product underwent electrophoresis on a 2% Agarose I™ gel before staining with Diamond™ Nucleic Acid Dye and imaging.

### REFERENCES FOR SUPPLEMENTAL TEXT

### References for Example 1

1. J. Kwon, S. Namgoong, N. H. Kim, CRISPR/Cas9 as Tool for Functional Study of Genes Involved in Preimplantation Embryo Development. Plos One 10 (2015).
2. O. C. Ikonomov et al., The Phosphoinositide Kinase PIKfyve Is Vital in Early Embryonic Development PREIMPLANTATION LETHALITY OF PIKfyve(−/−) EMBRYOS BUT NORMALITY OF PIKfyve(+/−) MICE. J Biol Chem 286, 13404-13413 (2011).
3. Y. Xiao et al., Regulation of gene expression in the bovine blastocyst by colony-stimulating factor 2 is disrupted by CRISPR/Cas9-mediated deletion of CSF2RA. Biology of Reproduction 104, 995-1007 (2021).
4. P. Stamatiadis et al., TEAD4 regulates trophectoderm differentiation upstream of CDX2 in a GATA3-independent manner in the human preimplantation embryo. Human Reproduction 37, 1760-1773 (2022).
5. G. Cosemans et al., CRISPR/Cas9 mediated knock-out (KO) reveals a divergent role for trophectoderm markers GATA2/3 in the mouse and human preimplantation embryo. Human Reproduction 37, 1128-1128 (2022).
6. Z. C. Tu, W. L. Yang, S. Yan, X. Y. Guo, X. J. Li, CRISPR/Cas9: a powerful genetic engineering tool for establishing large animal models of neurodegenerative diseases. Mol Neurodegener 10 (2015).
7. B. M. Motta, P. P. Pramstaller, A. A. Hicks, A. Rossini, The Impact of CRISPR/Cas9 Technology on Cardiac Research: From Disease Modelling to Therapeutic Approaches. Stem Cells Int 2017 (2017).
8. C. B. A. Whitelaw, Engineering Large Animal Models of Human Disease. J Pathol 240, 5-5 (2016).
9. E. Marr, C. J. Potter, Base Editing of Somatic Cells Using CRISPR-Cas9 in Drosophila. CRISPR J 4, 836-845 (2021).
10. C. Xie et al., Genome editing with CRISPR/Cas9 in postnatal mice corrects PRKAG2 cardiac syndrome. Cell Res 26, 1099-1111 (2016).
11. L. Swiech et al., In vivo interrogation of gene function in the mammalian brain using CRISPR-Cas9. Nat Biotechnol 33, 102-106 (2015).
12. M. Vilarino et al., CRISPR/Cas9 microinjection in oocytes disables pancreas development in sheep. Sci Rep 7, 17472 (2017).
13. A. H. Sergio Navarro-Serna, Analuce Canha-Gouveia, Ali Hanbashi, Gabriela Garrappa, Jordana S. Lopes, Evelyne Paris-Oller, Lucia Sarrias-Gil, Cesar Flores-Flores, Andrew Bassett, Raul Sanchez, Pablo Bermejo-Alvarez, Carmen Maths, Raquel Romar, John Parrington, Joaquin Gadea, Generation of Nonmosaic, Two-Pore Channel 2 Biallelic Knockout Pigs in One Generation by CRISPR-Cas9 Microinjection Before Oocyte Insemination. The CRISPR Journal 4 (2021).
14. I. L. Delerue F, Generation of Genetically Modified Mice through the Microinjection of Oocytes. Journal of Visualized Experiments doi: 10.3791/55765 (2017).
15. G. M. Gim et al., Germline transmission of MSTN knockout cattle via CRISPR-Cas9. Theriogenology 192, 22-27 (2022).
16. V. Morin, N. Veron, C. Marcelle, CRISPR/Cas9 in the Chicken Embryo. Methods Mol Biol 1650, 113-123 (2017).
17. B. Wang et al., Highly efficient CRISPR/HDR-mediated knock-in for mouse embryonic stem cells and zygotes. Biotechniques 59, 201-202, 204, 206-208 (2015).
18. J. R. Owen et al., One-step generation of a targeted knock-in calf using the CRISPR-Cas9 system in bovine zygotes. BMC Genomics 22, 118 (2021).
19. J. Ryu, K. Lee, CRISPR/Cas9-Mediated Gene Targeting during Embryogenesis in Swine. Methods Mol Biol 1605, 231-244 (2017).
20. Y. Song et al., Efficient dual sgRNA-directed large gene deletion in rabbit with CRISPR/Cas9 system. Cell Mol Life Sci 73, 2959-2968 (2016).
21. Z. Zhu, N. Verma, F. Gonzalez, Z. D. Shi, D. Huangfu, A CRISPR/Cas-Mediated Selection-free Knockin Strategy in Human Embryonic Stem Cells. Stem Cell Reports 4, 1103-1111 (2015).
22. H. Wang et al., One-step generation of mice carrying mutations in multiple genes by CRISPR/Cas-mediated genome engineering. Cell 153, 910-918 (2013).
23. T. Hai, F. Teng, R. Guo, W. Li, Q. Zhou, One-step generation of knockout pigs by zygote injection of CRISPR/Cas system. Cell Res 24, 372-375 (2014).
24. H. Wan et al., One-step generation of p53 gene biallelic mutant Cynomolgus monkey via the CRISPR/Cas system. Cell Res 25, 258-261 (2015).
25. K. E. Park et al., One-Step Homology Mediated CRISPR-Cas Editing in Zygotes for Generating Genome Edited Cattle. CRISPR J 3, 523-534 (2020).
26. M. Jinek et al., A programmable dual-RNA-guided DNA endonuclease in adaptive bacterial immunity. Science 337, 816-821 (2012).
27. L. P. B.-D. Arildo Nerys-Junior, Paula Pezzuto, Vinicius Cotta-de-Almeida, Amilcar Tanuri Comparison of the editing patterns and editing efficiencies of TALEN and CRISPR-Cas9 when targeting the human CCR5 gene. Genetics and Molecular Biology (2018).
28. Z. He, C. Proudfoot, C. B. Whitelaw, S. G. Lillico, Comparison of CRISPR/Cas9 and TALENs on editing an integrated EGFP gene in the genome of HEK293FT cells. Springerplus 5, 814 (2016).
29. J. Zhang et al., Comparison of gene editing efficiencies of CRISPR/Cas9 and TALEN for generation of MSTN knock-out cashmere goats. Theriogenology 132, 1-11 (2019).
30. C. L. Hui LIU, Yu-hang ZHAO, Xue-jie HAN, Zheng-wei ZHOU, Chen WANG, Rong-feng LI, Xue-ling LI, Comparing successful gene knock-in efficiencies of CRISPR/Cas9 with ZFNs and TALENs gene editing systems in bovine and dairy goat fetal fibroblasts. Journal of Integrative Agriculture 17 (2018).
31. P. D. Vos, A. Filipovska, O. Rackham, Frankenstein Cas9: engineering improved gene editing systems. Biochem Soc Trans 50, 1505-1516 (2022).
32. I. Lamas-Toranzo et al., Strategies to reduce genetic mosaicism following CRISPR-mediated genome edition in bovine embryos. Sci Rep 9, 14900 (2019).
33. D. Miskel et al., The cell cycle stage of bovine zygotes electroporated with CRISPR/Cas9-RNP affects frequency of Loss-of-heterozygosity editing events. Sci Rep 12, 10793 (2022).
34. J. C. Lin, A. L. Van Eenennaam, Electroporation-Mediated Genome Editing of Livestock Zygotes. Front Genet 12, 648482 (2021).
35. L. S. A. Camargo, J. R. Owen, A. L. Van Eenennaam, P. J. Ross, Efficient One-Step Knockout by Electroporation of Ribonucleoproteins Into Zona-Intact Bovine Embryos. Frontiers in Genetics 11 (2020).
36. B. W. Daigneault, S. Rajput, G. W. Smith, P. J. Ross, Embryonic POU5F1 is Required for Expanded Bovine Blastocyst Formation. Sci Rep-Uk 8 (2018).
37. D. Miao, M. I. Giassetti, M. Ciccarelli, B. Lopez-Biladeau, J. M. Oatley, Simplified pipelines for genetic engineering of mammalian embryos by CRISPR-Cas9 electroporationdagger. Biol Reprod 101, 177-187 (2019).
38. B. W. Daigneault, S. Rajput, G. W. Smith, P. J. Ross, Embryonic POU5F1 is Required for Expanded Bovine Blastocyst Formation. Sci Rep-Uk 8, 7753 (2018).
39. Z. Namula et al., Genome mutation after the introduction of the gene editing by electroporation of Cas9 protein (GEEP) system into bovine putative zygotes. In Vitro Cellular & Developmental Biology—Animal 55, 598-603 (2019).
40. Z. Namula et al., Genome mutation after the introduction of the gene editing by electroporation of Cas9 protein (GEEP) system into bovine putative zygotes. In Vitro Cell Dev Biol Anim 55, 598-603 (2019).
41. Z. Namula et al., Zona pellucida treatment before CRISPR/Cas9-mediated genome editing of porcine zygotes. Veterinary Medicine and Science 8, 164-169 (2022).
42. M. Mourot et al., The influence of follicle size, FSH-enriched maturation medium, and early cleavage on bovine oocyte maternal mRNA levels. Mol Reprod Dev 73, 1367-1379 (2006).
43. T. Fair et al., Analysis of differential maternal mRNA expression in developmentally competent and incompetent bovine two-cell embryos. Mol Reprod Dev 67, 136-144 (2004).
44. Q.-Q. Sha, J. Zhang, H.-Y. Fan, A story of birth and death: mRNA translation and clearance at the onset of maternal-to-zygotic transition in mammals†. Biology of Reproduction 101, 579-590 (2019).
45. K. Zhang, G. W. Smith, Maternal control of early embryogenesis in mammals. Reprod Fertil Dev 27, 880-896 (2015).
46. O. O. Abudayyeh et al., RNA targeting with CRISPR-Cas13. Nature 550, 280-284 (2017).
47. S. Kurosaka, S. Eckardt, K. J. McLaughlin, Pluripotent lineage definition in bovine embryos by Oct4 transcript localization. Biol Reprod 71, 1578-1582 (2004).
48. D. R. Khan et al., Expression of pluripotency master regulators during two key developmental transitions: EGA and early lineage specification in the bovine embryo. Plos One 7, e34110 (2012).
49. J. Nichols et al., Formation of pluripotent stem cells in the mammalian embryo depends on the POU transcription factor Oct4. Cell 95, 379-391 (1998).
50. N. M. E. Fogarty et al., Genome editing reveals a role for OCT4 in human embryogenesis. Nature 550, 67-73 (2017).
51. G. J. Pan, Z. Y. Chang, H. R. Scholer, D. Pei, Stem cell pluripotency and transcription factor Oct4. Cell Res 12, 321-329 (2002).
52. J. Sharma, M. Antenos, P. Madan, A Comparative Analysis of Hippo Signaling Pathway Components during Murine and Bovine Early Mammalian Embryogenesis. Genes (Basel) 12 (2021).
53. T. Frum et al., Oct4 cell-autonomously promotes primitive endoderm development in the mouse blastocyst. Dev Cell 25, 610-622 (2013).
54. G. Wu et al., Establishment of totipotency does not depend on Oct4A. Nat Cell Biol 15, 1089-1097 (2013).
55. C. Gerri et al., A conserved role of the Hippo signalling pathway in initiation of the first lineage specification event across mammals. Development 150 (2023).
56. K. Simmet et al., OCT4/POU5F1 is required for NANOG expression in bovine blastocysts. P Natl Acad Sci USA 115, 2770-2775 (2018).
57. K. Simmet et al., OCT4/POU5F1 is indispensable for the lineage differentiation of the inner cell mass in bovine embryos. Faseb Journal 36 (2022).
58. K. Simmet et al., OCT4/POU5F1 is indispensable for the lineage differentiation of the inner cell mass in bovine embryos. The FASEB Journal 36, e22337 (2022).
59. K. Simmet et al., OCT4/POU5F1 is required for NANOG expression in bovine blastocysts. Proceedings of the National Academy of Sciences 115, 2770-2775 (2018).
60. A. Korablev, V. Lukyanchikova, I. Serova, N. Battulin, On-Target CRISPR/Cas9 Activity Can Cause Undesigned Large Deletion in Mouse Zygotes. International Journal of Molecular Sciences 21 (2020).
61. D. D. G. Owens et al., Microhomologies are prevalent at Cas9-induced larger deletions. Nucleic Acids Res 47, 7402-7417 (2019).
62. F. A. Ran et al., Double nicking by RNA-guided CRISPR Cas9 for enhanced genome editing specificity. Cell 154, 1380-1389 (2013).
63. A. Korablev, V. Lukyanchikova, I. Serova, N. Battulin, On-Target CRISPR/Cas9 Activity Can Cause Undesigned Large Deletion in Mouse Zygotes. Int J Mol Sci 21 (2020).
64. S. Ye, B. Enghiad, H. Zhao, E. Takano, Fine-tuning the regulation of Cas9 expression levels for efficient CRISPR-Cas9 mediated recombination in Streptomyces. J Ind Microbiol Biotechnol 47, 413-423 (2020).
65. O. O. Abudayyeh et al., C2c2 is a single-component programmable RNA-guided RNA-targeting CRISPR effector. Science 353, aaf5573 (2016).
66. D. B. T. Cox et al., RNA editing with CRISPR-Cas13. Science 358, 1019-1027 (2017).
67. D. Bi et al., CRISPR/Cas13d-mediated efficient KDM5B mRNA knockdown in porcine somatic cells and parthenogenetic embryos. Reproduction 162, 149-160 (2021).
68. Y. Ai, D. Liang, J. E. Wilusz, CRISPR/Cas13 effectors have differing extents of off-target effects that limit their utility in eukaryotic cells. Nucleic Acids Res 50, e65 (2022).
69. M. Scarola et al., Epigenetic silencing of Oct4 by a complex containing SUV39H1 and Oct4 pseudogene lncRNA. Nature Communications 6 (2015).
70. H. Lin, A. Shabbir, M. Molnar, T. Lee, Stem cell regulatory function mediated by expression of a novel mouse Oct4 pseudogene. Biochemical and Biophysical Research Communications 355, 111-116 (2007).
71. S. Liedtke, J. Enczmann, S. Waclawczyk, P. Wernet, G. Kogler, Oct4 and its pseudogenes confuse stem cell research. Blood 110, 1081a-1081a (2007).
72. P. S. Yadav, W. A. Kues, D. Herrmann, J. W. Carnwath, H. Niemann, Bovine ICM derived cells express the Oct4 ortholog. Mol Reprod Dev 72, 182-190 (2005).
73. N. A. Salem et al., A novel Oct4/Pou5f1-like non-coding RNA controls neural maturation and mediates developmental effects of ethanol. Neurotoxicol Teratol 83, 106943 (2021).
74. A. Schiffmacher, C. Keefer, Expression of an OCT4 (POUSF1) pseudogene in the bovine embryo derived CT-1 cell line. Biol Reprod, 100-100 (2008).
75. E. Z. Sim et al., Methionine metabolism regulates pluripotent stem cell pluripotency and differentiation through zinc mobilization. Cell Rep 40, 111120 (2022).
76. N. Shiraki et al., Methionine metabolism regulates maintenance and differentiation of human pluripotent stem cells. Cell Metab 19, 780-794 (2014).
77. C. Huang, P. Santofimia-Castano, J. Iovanna, NUPR1: A Critical Regulator of the Antioxidant System. Cancers (Basel) 13 (2021).
78. B. S. Mallon et al., StemCellDB: the human pluripotent stem cell database at the National Institutes of Health. Stem Cell Res 10, 57-66 (2013).
79. R. Raz, C. K. Lee, L. A. Cannizzaro, P. d'Eustachio, D. E. Levy, Essential role of STAT3 for embryonic stem cell pluripotency. P Natl Acad Sci USA 96, 2846-2851 (1999).
80. T. Ripich et al., SWEF Proteins Distinctly Control Maintenance and Differentiation of Hematopoietic Stem Cells. PLoS One 11, e0161060 (2016).
81. Z. Luo et al., Zic2 is an enhancer-binding factor required for embryonic stem cell specification. Mol Cell 57, 685-694 (2015).
82. L. S. Lim et al., Zic3 is required for maintenance of pluripotency in embryonic stem cells. Molecular Biology of the Cell 18, 1348-1358 (2007).
83. R. A. Lea et al., KLF17 promotes human naive pluripotency but is not required for its establishment. Development 148 (2021).
84. S. H. Wang et al., KLF17 promotes human naive pluripotency through repressing MAPK3 and ZIC2. Sci China Life Sci 65, 1985-1997 (2022).
85. W. K. Hua et al., HDAC8 regulates long-term hematopoietic stem-cell maintenance under stress by modulating p53 activity. Blood 130, 2619-2630 (2017).
86. R. Y. Tsai, R. D. McKay, A nucleolar mechanism controlling cell proliferation in stem cells and cancer cells. Genes Dev 16, 2991-3003 (2002).
87. C. M. V. Barbosa et al., Extracellular annexin-A1 promotes myeloid/granulocytic differentiation of hematopoietic stem/progenitor cells via the Ca(2+)/MAPK signalling transduction pathway. Cell Death Discov 5, 135 (2019).
88. N. N. Kreis, F. Louwen, J. Yuan, The Multifaceted p21 (Cip1/Waf1/CDKN1A) in Cell Differentiation, Migration and Cancer Therapy. Cancers (Basel) 11 (2019).
89. L. L. Tran, T. Dang, R. Thomas, D. R. Rowley, ELF3 mediates IL-lalpha induced differentiation of mesenchymal stem cells to inflammatory iCAFs. Stem Cells 39, 1766-1777 (2021).
90. Y. J. Xie et al., Leucine-Rich Glioma Inactivated 1 Promotes Oligodendrocyte Differentiation and Myelination via TSC-mTOR Signaling. Front Mol Neurosci 11, 231 (2018).
91. J. Laurincik, V. Kopecny, P. Hyttel, Detailed analysis of pronucleus development in bovine zygotes in vivo: ultrastructure and cell cycle chronology. Mol Reprod Dev 43, 62-69 (1996).
92. J. Laurincik et al., A detailed analysis of pronucleus development in bovine zygotes in vitro: cell-cycle chronology and ultrastructure. Mol Reprod Dev 50, 192-199 (1998).
93. P. Comizzoli, B. Marquant-Le Guienne, Y. Heyman, J. P. Renard, Onset of the first S-phase is determined by a paternal effect during the G1-phase in bovine zygotes. Biol Reprod 62, 1677-1684 (2000).
94. M. A. M. Jada Nix, Mary Ali Oliver, Michelle Rhoads, Alan D. Ealy, Fernando H. Biase, Cleavage kinetics is a better indicator of embryonic developmental competency than brilliant cresyl blue staining of oocytes. Anim Reprod Sci (2023).
95. P. Tribulo, R. M. Rivera, M. S. Ortega Obando, E. A. Jannaman, P. J. Hansen, Production and Culture of the Bovine Embryo. Methods Mol Biol 2006, 115-129 (2019).
96. J. P. Concordet, M. Haeussler, CRISPOR: intuitive guide selection for CRISPR/Cas9 genome editing experiments and screens. Nucleic Acids Res 46, W242-W245 (2018).
97. H. H. Wessels et al., Massively parallel Cas13 screens reveal principles for guide RNA design. Nature Biotechnology 38, 722-+(2020).
98. X. Guo et al., Transcriptome-wide Cas13 guide RNA design for model organisms and viral RNA pathogens. Cell Genom 1 (2021).
99. W. J. Kent et al., The human genome browser at UCSC. Genome Res 12, 996-1006 (2002).
100. S. Karmakar, D. Behera, M. J. Baig, K. A. Molla, “In Vitro Cas9 Cleavage Assay to Check Guide RNA Efficiency” in CRISPR-Cas Methods: Volume 2, M. T. Islam, K. A. Molla, Eds. (Springer US, New York, NY, 2021), 10.1007/978-1-0716-1657-4_3, pp. 23-39.
101. K. Clement et al., CRISPResso2 provides accurate and rapid genome editing sequence analysis. Nature Biotechnology 37, 224-226 (2019).
102. A. M. Bolger, M. Lohse, B. Usadel, Trimmomatic: a flexible trimmer for Illumina sequence data. Bioinformatics 30, 2114-2120 (2014).
103. W. Shen, S. Le, Y. Li, F. Q. Hu, SeqKit: A Cross-Platform and Ultrafast Toolkit for FASTA/Q File Manipulation. Plos One 11 (2016).
104. R. R. Wick, L. M. Judd, K. E. Holt, Performance of neural network basecalling tools for Oxford Nanopore sequencing. Genome Biol 20, 129 (2019).
105. H. Li, Minimap2: pairwise alignment for nucleotide sequences. Bioinformatics 34, 3094-3100 (2018).
106. H. Li et al., The Sequence Alignment/Map format and SAMtools. Bioinformatics 25, 2078-2079 (2009).
107. G. B6, R. Mapletoft, Evaluation and classification of bovine embryos. Animal Reproduction (AR) 10, 344-348 (2018).
108. F. H. Biase, M. M. Franco, L. R. Goulart, R. C. Antunes, Protocol for extraction of genomic DNA from swine solid tissues. Genet Mol Biol 25, 313-315 (2002).
109. F. H. Biase, Isolation of high-quality total RNA and RNA sequencing of single bovine oocytes. STAR Protoc 2, 100895 (2021).
110. C. Puissant, L. M. Houdebine, An Improvement of the Single-Step Method of Rna Isolation by Acid Guanidinium Thiocyanate-Phenol-Chloroform Extraction. Biotechniques 8, 148-149 (1990).
111. P. Chomczynski, A Reagent for the Single-Step Simultaneous Isolation of Rna, DNA and Proteins from Cell and Tissue Samples. Biotechniques 15, 532-& (1993).
112. P. Chomczynski, N. Sacchi, The single-step method of RNA isolation by acid guanidinium thiocyanate-phenol-chloroform extraction: twenty-something years on. Nat Protoc 1, 581-585 (2006).
113. J. W. Bagnoli et al., Sensitive and powerful single-cell RNA sequencing using mcSCRB-seq. Nature Communications 9, 2937 (2018).
114. C. G. Elsik et al., The genome sequence of taurine cattle: a window to ruminant biology and evolution. Science 324, 522-528 (2009).
115. R. J. Kinsella et al., Ensembl BioMarts: a hub for data retrieval across taxonomic space. Database 2011, bar030 (2011).
116. P. Flicek et al., Ensembl 2012. Nucleic Acids Res 40, D84-90 (2012).
117. D. Kim, J. M. Paggi, C. Park, C. Bennett, S. L. Salzberg, Graph-based genome alignment and genotyping with HISAT2 and HISAT-genotype. Nat Biotechnol 37, 907-915 (2019).
118. P. Danecek et al., Twelve years of SAMtools and BCFtools. Gigascience 10 (2021).
119. G. Tischler, S. Leonard, biobambam: tools for read pair collation based algorithms on BAM files. Source Code Biol Med 9 (2014).
120. Y. Liao, G. K. Smyth, W. Shi, featureCounts: an efficient general purpose program for assigning sequence reads to genomic features. Bioinformatics 30, 923-930 (2014).
121. J. Fox, S. Weisberg, An R Companion to Applied Regression (Sage, Thousand Oaks CA, ed. Third, 2019).
122. M. Hollander, D. A. Wolfe, E. Chicken, Nonparametric statistical methods (John Wiley & Sons, 2013).
123. R. Ihaka, R. Gentleman, R: A Language for Data Analysis and Graphics. J Comput Graph Stat 5, 299-314 (1996).
124. RCoreTeam (2020) R: A Language and Environment for Statistical Computing. (R Foundation for Statistical Computing, Vienna, Austria).
125. D. J. McCarthy, G. K. Smyth, edgeR: a Bioconductor package for differential expression analysis of digital gene expression data. Bioinformatics 26, 139-140 (2010).
126. B. Li, C. N. Dewey, RSEM: accurate transcript quantification from RNA-Seq data with or without a reference genome. BMC Bioinformatics 12, 323 (2011).
127. G. P. Wagner, K. Kin, V. J. Lynch, Measurement of mRNA abundance using RNA-seq data: RPKM measure is inconsistent among samples. Theory Biosci 131, 281-285 (2012).
128. M. I Love, W. Huber, S. Anders, Moderated estimation of fold change and dispersion for RNA-seq data with DESeq2. Genome Biol 15, 550 (2014).
129. Y. Benjamini, Y. Hochberg, Controlling the false discovery rate—a practical and powerful approach to multiple testing. J Roy Stat Soc B Met 57, 289-300 (1995).
130. T. R. Talluri et al., Derivation and characterization of bovine induced pluripotent stem cells by transposon-mediated reprogramming. Cell Reprogram 17, 131-140 (2015).
131. T. Kawaguchi et al., Generation of Naive Bovine Induced Pluripotent Stem Cells Using PiggyBac Transposition of Doxycycline-Inducible Transcription Factors. PLoS One 10, e0135403 (2015).
132. M. Khateb et al., Transcriptomics, regulatory syntax, and enhancer identification in mesoderm-induced ESCs at single-cell resolution. Cell Rep 40, 111219 (2022).
133. W. J. Kent, BLAT—the BLAST-like alignment tool. Genome research 12, 656-664 (2002).
134. R. M. Kuhn, D. Haussler, W. J. Kent, The UCSC genome browser and associated tools. Briefings in bioinformatics 14, 144-161 (2013).
135 B. J. Raney et al., Track data hubs enable visualization of user-defined genome-wide annotations on the UCSC Genome Browser. Bioinformatics (Oxford, England) 30, 1003-1005 (2014).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided.

What is claimed is:

1. A method of biallelic DNA modification comprising:
modifying a target DNA using a DNA modification agent; and
knocking down target RNA using an RNA modification agent.

2. The method of claim 1, wherein the DNA modification agent is a CRISPR-Cas system, a TALEN, a Zinc Finger Nuclease, an Omega System, a transposase, a recombinase, a recombinase-based system, a non-LTR retrotransposon, or a meganuclease.

3. The method of claim 1, wherein the DNA modification agent is a Type II CRISPR-Cas system.

4. The method of claim 3, wherein the DNA modification agent is a CRISPR-Cas9 system.

5. The method of claim 4, wherein the DNA modification agent is a CRISPR-Cas9D1-A system.

6. The method of claim 1, wherein the RNA modification agent is a CRISPR-Cas system or RNAi.

7. The method of claim 6, wherein the RNA modification agent is a Type VI CRISPR-Cas system.

8. The method of claim 7, wherein the RNA modification agent is a CRISPR-Cas13 system.

9. The method of claim 8, wherein the RNA modification agent is a CRISPR-Cas13a system.

10. The method of claim 1, wherein modifying, knocking down, or both occurs in vivo or in vitro.

11. The method of claim 10, wherein modifying, knocking down, or both occurs in a cell.

12. The method of claim 11, wherein the cell is a zygote.

13. The method of claim 11, wherein the cell is a non-human animal cell.

14. The method of claim 13, wherein the non-human animal is a bovine, porcine, ovine, canine, feline, or equine.

15. The method of claim 10, wherein delivery of the DNA modifying agent and/or RNA modifying agent occurs via electroporation.

16. A modified DNA produced by the method of claim 1.

17. A cell comprising modified DNA produced by the method of claim 1.

18. The cell of claim 17, wherein the cell is a zygote.

19. The cell of claim 17, wherein the cell is a non-human animal cell.

20. The cell of any one of claim 19, wherein the non-human animal is a bovine, porcine, ovine, canine, feline, or equine.
